SEQUENCE LISTING (6 sequences)

SEQ ID NO: 1 - DNA, 46 bp, synthetic construct
  source 1..46: mol_type = other DNA
cgatcatctcagaacattcttagcgttttgttcttgtgtatgatcg

SEQ ID NO: 2 - DNA, 16 bp, synthetic construct
  source 1..16: mol_type = other DNA
gggtgggttgggaggg

SEQ ID NO: 3 - DNA, 66 bp, synthetic construct
  source 1..66: mol_type = other DNA
atagtctatcgatcaattgggtctgggtccgtgccggcgtggtcttgtagatagcaagtgtattca

SEQ ID NO: 4 - protein, 949 aa, synthetic construct
MSAMTKGLRNCKGCVNMKKKMSLREKREAEKQAKKAAYSAASKNTDSKPAEKKAETPKPAEIISDNSRNKTAVKAAGLKSTIISGDKLYMTSFGKGNAAVIEQKIDINDYSFSAMKDTPSLEVDKAESKEISFSSHHPFVKNDKLTTYNPLYGGKDNPEKPVGRDMLGLKDKLEERYFGCTFNDNLHIQIIYNILDIEKILAVHSANITTALDHMVDEDDEKYLNSDYIGYMNTINTYDVFMDPSKNSSLSPKDRKNIDNSRAKFEKLLSTKRLGYFGFDYDANGKDKKKNEEIKKRLYHLTAFAGQLRQWSFHSAGNYPRTWLYKLDSLDKEYLDTLDHYFDKRFNDINDDFVTKNATNLYILKEVFPEANFKDIADLYYDFIVIKSHKNMGFSIKKLREKMLECDGADRIKEQDMDSVRSKLYKLIDFCIFKYYHEFPELSEKNVDILRAAVSDTKKDNLYSDEAARLWSIFKEKFLGFCDKIVVWVTGEHEKDITSVIDKDAYRNRSNVSYFSKLMYAMCFFLDGKEINDLLTTLINKFDNIANQIKTAKELGINTAFVKNYDFFNHSEKYVDELNIVKNIARMKKPSSNAKKAMYHDALTILGIPEDMDEKALDEELDLILEKKTDPVTGKPLKGKNPLRNFIANNVIENSRFIYLIKFCNPENVRKIVNNTKVTEFVLKRIPDAQIERYYKSCTDSEMNPPTEKKITELAGKLKDMNFGNFRNVRQSAKENMEKERFKAVIGLYLTVVYRVVKNLVDVNSRYIMAFHSLERDSQLYNVSVDNDYLALTDTLVKEGDNSRSRYLAGNKRLRDCVKQDIDNAKKWFVSDKYNSITKYRNNVAHLTAVRNCAEFIGDITKIDSYFALYHYLIQRQLAKGLDHERSGFDRNYPQYAPLFKWHTYVKDVVKALNAPFGYNIPRFKNLSIDALFDRNEIKKNDGEKKSDD

SEQ ID NO: 5 - RNA, 59 nt, synthetic construct
  source 1..59: mol_type = other RNA
gaactatagtagtgtaaatttacactactctaaaactcctaaaattcccttagtcagag

SEQ ID NO: 6 - RNA, 59 nt, synthetic construct
  source 1..59: mol_type = other RNA
tcctaaaattcccttagtcagaggaactatagtagtgtaaatttacactactctaaaac

(19) United States
(12) Patent Application Publication
(10) Pub. No.: US 2025/0223659 A1
(43) Pub. Date: Jul. 10, 2025

(54) METHODS AND USES OF CRISPR CASCADE REACTIONS FOR CRISPR DIAGNOSTICS

(71) Applicant: Roche Molecular Systems, Inc., Pleasanton, CA (US)

(72) Inventors: Xiang Li, Pleasanton, CA (US); Yu Tian, Pleasanton, CA (US)

(73) Assignee: Roche Molecular Systems, Inc., Pleasanton, CA (US)

(21) Appl. No.: 19/013,817

(22) Filed: Jan. 8, 2025

Related U.S. Application Data
(60) Provisional application No. 63/618,636, filed on Jan. 8, 2024.

(51) Int. Cl. C12Q 1/70; C12Q 1/28; C12Q 1/44; C12Q 1/48; C12Q 1/6823; C12Q 1/6825

(52) U.S. Cl. CPC C12Q 1/703; C12Q 1/28; C12Q 1/44; C12Q 1/485; C12Q 1/6823; C12Q 1/6825; C12Q 1/708; G01N 2333/025; G01N 2333/11; G01N 2333/16; G01N 2333/165; G01N 2333/90241; G01N 2333/908; G01N 2333/9126; G01N 2333/922

(57) ABSTRACT

The present disclosure relates to enzymatic cascade reactions for achieving better sensitivity in detecting a target nucleic acid sequence. Several aspects of the disclosure relate to a reaction master mix comprising four major components: a CRISPR enzyme, a guide RNA (gRNA), an aptamer (e.g., an inhibiting aptamer or an activating aptamer) and a “signaling” (i.e., reporter) enzyme. The aptamer interacts with the signaling enzyme and forms a complex, resulting in, e.g., inhibited signaling enzyme activity. When a target is present in the reaction mix, the Cas/guideRNA system becomes activated and preferentially collaterally cleave(s) all nucleic acids in the solution, including the aptamer. Once aptamer gets cleaved, the signaling enzyme is free to produce a signal. In the presence of a substrate, such signaling enzyme activity produces a robust signal that significantly improves the limit-of-detection of other techniques in the art.

### RELATED APPLICATIONS

This application claims priority under 35 U.S.C. 119 (e) to U.S. Provisional Patent Application Ser. No. 63/618,636, filed 8 Jan. 2024 and titled “Methods And Uses of CRISPR Cascade Reactions For CRISPR Diagnostics,” the disclosure of which is hereby incorporated by reference in its entirety.

### SEQUENCE LISTING

The official copy of the sequence listing is submitted electronically via EFS-Web with a file named, “08485-007US1.xml,” created on Jan. 7, 2025, and having a size of 7 kilobytes, and is filed concurrently with the specification. The sequence listing contained in the XML formatted document is part of the specification and is herein incorporated by reference in its entirety.

### FIELD

The present invention relates to CRISPR.

### BACKGROUND

The activity of an enzyme may be controlled by an interaction with an aptamer. Aptamers, like, e.g., peptides generated by phage display or monoclonal antibodies, are capable of specifically binding to selected targets, e.g., enzymes, and modulating the target's activity or binding interactions. For example, through binding its target, an aptamer may inhibit or activate the target's ability to function. Typically identified by an in vitro selection process from pools of random sequence oligonucleotides, aptamers have been generated for hundreds of proteins, including growth factors, transcription factors, enzymes, immunoglobulins and receptors. A typical aptamer is 10-15 kDa in size (20-45 nucleotides), binds its target with nanomolar to sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarities, hydrophobic contacts and steric exclusion) that drive affinity and specificity in antibody-antigen complexes.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

A CRISPR system can cleave an aptamer complexed to an enzyme, e.g., a reporter enzyme, when the CRISPR system is activated in the presence of a target polynucleotide sequence (target sequence). Such cleavage can modulate the activity of the enzyme. For example, where the enzyme is a reporter enzyme, such cleavage can directly or indirectly activate or inhibit production of a detectable signal with improved sensitivity towards RNA targets or DNA targets. The present disclosure provides a technical improvement that significantly overcomes shortcomings of the state-of-art in detecting a target nucleic acid sequence using CRISPR.

In some aspects, provided herein are one or more reaction mixture(s) comprising a CRISPR enzyme (also referred to herein as a CRISPR-Cas enzyme or CRISPR nuclease) with nuclease activity and a guide RNA (also referred to herein as a guide nucleic acid or gRNA), wherein the gRNA is capable of forming a CRISPR system with the CRISPR enzyme and a region of the gRNA is complimentary to a pre-determined target polynucleotide acid sequence. The CRISPR enzyme and gRNA may be in a CRISPR system comprising, consisting essentially of, or consisting of: the CRISPR enzyme and the gRNA, or the CRISPR enzyme and gRNA may be initially separate and then form such CRISPR system in the reaction mixture. The reaction mixture further includes a second enzyme, e.g., a reporter enzyme, and a nucleic acid aptamer capable of binding to the second enzyme. The second enzyme and the aptamer may be in a complex comprising the second enzyme and the aptamer and/or the aptamer and enzyme may be initially separate and then form such complex in the reaction mixture. The reaction mixture may further include a substrate for the second enzyme, e.g., for the reporter enzyme. The reaction mixture may further include the target polynucleotide sequence, which may be obtained from or present in a biological sample. In the presence of a target polynucleotide sequence, e.g., when region of the gRNA is hybridized to a pre-determined target polynucleotide acid sequence, the CRISPR enzyme of the CRISPR system can cleave the aptamer while the aptamer is bound to the second enzyme.

The nucleic acid aptamer can be an inhibitor, an activator, or it can otherwise regulate (modulate) the activity of the second enzyme when in a complex with the second enzyme. For example, if the nucleic acid aptamer in a complex with the second enzyme is an inhibitor of the second enzyme, the second enzyme, upon cleavage of the nucleic acid aptamer by an activated CRISPR enzyme, becomes free to act upon its substrate. If the aptamer is an inhibiting aptamer, e.g., an inhibitor of a reporter enzyme, the reporter enzyme, upon cleavage of the inhibiting aptamer, becomes free to directly or indirectly produce a detectable signal, e.g., by action of the reporter enzyme on its substrate. In embodiments in which the aptamer is an inhibitor of a reporter enzyme, the detectable signal directly or indirectly produced by the activity of the reporter enzyme on its substrate following cleavage of the aptamer can be substantially higher than a detectable signal obtained by cleaving a nucleic acid that does not modulate the activity of a reporter enzyme, e.g., a detectable signal by cleaving a nucleic acid attached to a fluorescent probe wherein the cleavage does not modulate the activity of a reporter enzyme.

Alternatively, if the nucleic acid aptamer in a complex with the second enzyme is an activating aptamer, e.g., an activator of the second enzyme, the second enzyme, upon cleavage of the activating aptamer by an activated CRISPR enzyme, exhibits reduced activity towards the substrate. For example, if the aptamer is an activator of a reporter enzyme, the reporter enzyme, upon cleavage of the aptamer, decreases a rate activity towards its substrate thereby directly or indirectly leading to a change in a detectable signal, e.g., a decreased detectable signal. In embodiments in which the aptamer is activator of a reporter enzyme, a detectable signal, e.g., a relative or absolute magnitude of a change (decrease) in the detectable signal, directly or indirectly produced by the upon action of the reporter enzyme on its substrate following cleavage of the aptamer can be substantially greater than a detectable signal, e.g., a relative or absolute magnitude of a change (decrease) in the detectable signal, obtained by cleaving a nucleic acid that does not modulate the activity of a reporter enzyme.

In some aspects, the nucleic acid aptamer is 20 nucleotides to 100 nucleotides in length, but any length suitable to form a complex with the second enzyme, e.g., the reporter enzyme, is contemplated. Preferably, the nucleic acid aptamer binds to the second enzyme, e.g., the reporter enzyme and is cleaved by the CRISPR enzyme once the CRISPR system becomes activated by a target polynucleotide sequence. The nucleic acid aptamer can be a single stranded deoxyribonucleic acid (DNA) molecule, a double stranded deoxyribonucleic acid (DNA) molecule, or a ribonucleic acid (RNA) molecule.

In any of the reaction mixtures, kits, methods and other embodiments disclosed herein, the reporter enzyme can be any enzyme capable of directly or indirectly producing a detectable signal, e.g., a change in the detectable signal, e.g., upon cleavage by a CRISPR nuclease of a nucleic acid aptamer in a complex with the reporter enzyme. For example, the reporter enzyme can be any enzyme capable of acting upon a substrate to directly or indirectly produce a detectable signal, e.g., a change in a detectable signal. Exemplary reporter enzymes suitable for use in any of the reaction mixtures, kits, methods and other embodiments disclosed herein include peroxidase, an oxidase, a phosphatase, a polymerase, a second CRISPR enzyme, or combinations thereof. For example, reporter enzymes can be selected from the group consisting of β-galactosidase, luciferase, alkaline phosphatase, horseradish peroxidase (HRP), β-glucuronidase (GUS), and combinations thereof. In some instances, the reporter enzyme is a DNA polymerase and the aptamer is an inhibiting aptamer for the DNA polymerase, e.g., the DNA polymerase can be a Z05 polymerase and the inhibiting aptamer can be a Z05 NTQ aptamer (5′-CGATCATCTCAGAACATTCTTAGCGTTTTGTTCTTGTGTATGATCG-3′; SEQ ID NO:1). In other instances, the reporter enzyme can be a peroxidase and the aptamer can be an activating aptamer for upregulating peroxidase activity, e.g., the peroxidase can be a horseradish peroxidase and the activating aptamer is a G-quaruplex (G4)-forming DNA aptamer (e.g., 5′-GGGTGGGTTGGGAGGG-3′; SEQ ID NO:2). In some instances, the reporter enzyme can be a luciferase and the aptamer can be an inhibiting aptamer for inhibiting luciferase activity (e.g., 5′-ATAGTCTATCGATCAATTGGGTCTGGGTCCGTGCCGGCGTGGTCTTGTAGATAGCAA GTGTATTCA-3′; SEQ ID NO:3).

In any of the reaction mixtures, kits, methods and other embodiments disclosed herein, the substrate for the reporter enzyme can be a substrate, e.g., an enzymatic substrate, able to directly or indirectly produce a detectable signal, e.g. a change in the detectable signal. Exemplary detectable signals produced in, used, or by any of the reaction mixtures, kits, methods, or other embodiments disclosed herein include an optical signal such as a fluorescence signal, phosphorescent signal, absorbance signal, colorimetric signal, chemiluminescent signal, or another suitable optical signal. Alternatively, or in combination therewith, the detectable signal may be a sensor signal such as an electrochemical signal or electrooptical signal. A detectable signal includes a change in such a signal, e.g., an increase or decrease thereof.

In any of the reaction mixtures, kits, methods and other embodiments disclosed herein, the target polynucleotide acid sequence can be a target sequence selected from any of the target sequences disclosed herein. For example, the target sequence may be from one or more pathogens such as a virus, e.g., one or more of Sars-COV-2, RSV, FluA, FluB, HIV, and human papilloma virus. In some embodiments, the CRISPR enzyme comprises trans-cleavage activity, e.g., a Cas 12 enzyme or a Cas13 enzyme. Certain CRISPR enzymes, e.g., Cas9 nucleases and Cas12 nucleases, are activated by the hybridization of a gRNA to a target polynucleotide sequence including a protospacer adjacent motif (PAM) or protospacer flanking sequence. Target polynucleotides of reaction mixtures, kits, methods, and other embodiments disclosed herein using such CRISPR enzymes typically include such protospacer sequences.

The reaction mixtures and systems described herein are characterized by the ability to detect a target sequence with a limit of detection at least as low as ˜10 fM (femtomolar). The reaction mixtures and systems described herein may be further characterized by sensitivities and specificities at least as high as for other techniques for detecting the presence or amount of a target polynucleotide such as PCR or isothermal polynucleotide amplification techniques, e.g., at least 90% or higher. In preferred cases, the reaction mixtures, methods, kits and systems described herein do not comprise or use any reagents for nucleic acid amplification. Methods using the reaction mixtures, kits, and systems described herein can be performed without substantially replicating, e.g., without replicating, or otherwise substantially amplifying, e.g., without amplifying, the target polynucleotide sequence and/or any polynucleotide sequences including the target polynucleotide sequence.

In some aspects, provided herein are kits comprising reagents and materials for detecting a target nucleic acid sequence. In some aspects the disclosure provides a kit comprising reagents including: a CRISPR enzyme; a gRNA wherein the gRNA is capable of forming a CRISPR system with the CRISPR enzyme and a region of the gRNA is complimentary to a pre-determined target nucleic acid sequence and wherein the gRNA is bound with the CRISPR enzyme in a CRISPR system or is a gRNA configured to form a CRISPR system with and including the CRISPR enzyme; a nucleic acid aptamer; and a second enzyme, e.g., a reporter enzyme, wherein the aptamer is complexed with the second enzyme, e.g., reporter enzyme, or is an aptamer configured to form a complex with the second enzyme, e.g., reporter enzyme; and a substrate for the second enzyme, e.g., reporter enzyme. The reagents of the kit, e.g., the CRISPR enzyme, gRNA, second enzyme, e.g., reporter enzyme, and substrate may independently be selected from any of such reagents disclosed herein. The target sequence to which a region of the gRNA of the kit is complementary may be any of the target sequences disclosed herein.

Such kits may include additional reagents or materials to facilitate use of the kit. Such additional reagents or materials may include, e.g., reagents and/or materials for preparing a biological sample or the foregoing reagents for detecting a target(s) in a sample and/or for performing the detection of such target(s), one or more liquid reagents such as a lysis buffer or wash buffer, one or more particles such as magnetic beads or polymer beads, co-factors associated with an enzyme of the kit, reagents suitable for extracting a plurality of nucleic acids from a biological sample.

Any of the reagents and materials of any of the kits disclosed herein may be disposed in or among one or more containers, e.g., one or more tube(s) or microfluidic device(s). Such reagents or materials may be present in liquid form, e.g., within a tube(s) or blister pack of a microfluidic device. Alternatively, or in combination with liquid reagent(s) or materials, one or more of the reagents may be in dry, e.g., lyophilized form, e.g., within a container such as a tube or microfluidic device. In some aspects, at least one container, e.g., a tube or a microfluidic device, comprises a filter for separating cellular debris disrupted from the lysis buffer from a plurality of nucleic acids from a biological sample. In some configurations, at least one container is a thermoplastic tube. In some configurations, at least one container is transparent.

In embodiments, a kit includes: one or more containers containing in or among them a CRISPR enzyme; a gRNA wherein the gRNA is capable of forming a CRISPR system with the CRISPR enzyme and a region of the gRNA is complimentary to a pre-determined target nucleic acid sequence and wherein the gRNA is bound with the CRISPR enzyme in a CRISPR system or is a gRNA configured to form a CRISPR system with and including the CRISPR enzyme; a nucleic acid aptamer; a second enzyme, e.g., a reporter enzyme, wherein the aptamer is complexed with the second enzyme, e.g., reporter enzyme, or is an aptamer configured to form a complex with the second enzyme, e.g., reporter enzyme; a substrate for the second enzyme, e.g., reporter enzyme; and a substrate for the second enzyme, e.g., reporter enzyme. The foregoing reagents of the kit may all be disposed within a single container or one or more of the reagents may be disposed within a first container separately from the other reagents in one or more second container(s). The reagents of the kit, e.g., the CRISPR enzyme, gRNA, second enzyme, e.g., reporter enzyme, and substrate may independently be selected from any of such reagents disclosed herein. The target sequence to which a region of the gRNA is complementary may be any of the target sequences disclosed herein.

Each container of any embodiments of the kit may independently be, e.g., a tube or a microfluidic device, e.g., a microfluidic channel, microfluidic chamber, liquid pouch or blister pack thereof. One or more containers of the kit may be formed of thermoplastic, e.g., as a thermoplastic tube. One or more containers of the kit may be transparent, e.g., a transparent tube. Reagents of the kit may be present in liquid form, e.g., in solution, and/or in dried, e.g., lyophilized form.

Any embodiments of the kit disclosed herein may include instructions for use thereof.

In some aspects, the disclosure provides a method for detecting a target nucleic acid sequence comprising: forming a reaction mixture comprising: a CRISPR system comprising a CRISPR enzyme with nuclease activity and a gRNA, wherein the gRNA is capable of forming the CRISPR system with the CRISPR enzyme and wherein a region of the gRNA is complimentary to a pre-determined target nucleic acid sequence; a complex of a nucleic acid aptamer and second enzyme, e.g., a reporter enzyme; a substrate the second enzyme, e.g., for the reporter enzyme; and a biological sample. The reagents of the method, e.g., the CRISPR enzyme, gRNA, second enzyme, e.g., reporter enzyme, and substrate may independently be selected from any of such reagents disclosed herein. The target sequence to which a region of the gRNA is complementary may be any of the target sequences disclosed herein.

Any of the reaction mixtures, kits, methods, or other embodiments disclosed herein may be configured for use with or use a biological sample that is or comprises, e.g., a nasal swab, a sputum sample, saliva, urine, a blood sample, e.g., whole blood, plasma, or serum, or another suitable sample for providing a plurality of nucleic acids to be tested for the presence of a target sequence. The reaction mixture may further include reagents to facilitate the detection of a target nucleic acid sequence, e.g., a label for producing a detectable signal, an enzyme co-factor(s), or liquid reagents such as a buffer.

In some aspects, the nucleic acid aptamer is an inhibiting aptamer, e.g., an inhibitor of the second enzyme, e.g., of the reporter enzyme. In other aspects, the nucleic acid aptamer is an activating aptamer, e.g., an activator of the second enzyme, e.g., reporter enzyme or otherwise regulates the activity thereof. In some aspects, the nucleic acid aptamer is 20 nucleotides to 100 nucleotides in length. In some aspects, the nucleic acid aptamer can bind to a corresponding second enzyme, e.g., reporter enzyme, and can be cleaved, e.g., while bound to the second enzyme, by nuclease activity of the CRISPR enzyme in the presence of a target polynucleotide sequence. In some aspects, the nucleic acid aptamer can be a single stranded deoxyribonucleic acid (DNA) molecule, a double stranded deoxyribonucleic acid (DNA) molecule, a ribonucleic acid (RNA) molecule, or another nucleic acid polymer. In some aspects, the second enzyme is a reporter enzyme selected from the group consisting of a peroxidase, an oxidase, a phosphatase, a polymerase, or a second CRISPR enzyme. In other aspects, the second enzyme, e.g., reporter enzyme, is a DNA polymerase and the aptamer is an inhibiting aptamer for the DNA polymerase. The DNA polymerase can be a Z05 polymerase and the inhibiting aptamer can be a Z05 NTQ aptamer. The reporter enzyme can be selected from the group consisting of β-galactosidase, luciferase, alkaline phosphatase, horseradish peroxidase (HRP), β-glucuronidase (GUS). In some instances, the reporter enzyme is a peroxidase and the aptamer is an activating aptamer for upregulating peroxidase activity. In some instances, the peroxidase is a horseradish peroxidase and the activating aptamer is a G-quaruplex (G4)-forming DNA aptamer. In some instances, the G-quaruplex (G4)-forming DNA aptamer comprises 5′-GGGTGGGTTGGGAGGG-3′ (SEQ ID NO:2). In some instances, the reporter enzyme is a luciferase and the aptamer is an inhibiting aptamer for inhibiting luciferase activity. In some instances, the inhibiting luciferase aptamer comprises 5′-ATAGTCTATCGATCAATTGGGTCTGGGTCCGTGCCGGCGTGGTCTTGTAGATAGCAA GTGTATTCA-3′ (SEQ ID NO:3).

In any of the reaction mixtures, kits, methods, or other embodiments disclosed herein, the substrate for the second enzyme, e.g., reporter enzyme, may directly or indirectly produce a detectable signal, e.g., an optical signal such as a florescence signal, a phosphorescent signal, a chemiluminescence signal, an absorbance signal, or a colorimetric signal, or an electrochemical signal. In some instances, the substrate for the reporter enzyme directly or indirectly produces a detectable signal, e.g., a change in the detectable signal, following activity of the reporter enzyme thereon. For example, the substrate may be non-fluorescent (fluorescent) prior to activity of the reporter enzyme thereon and fluorescent (non-fluorescent) thereafter. As another example, following activity of the reporter enzyme thereon, the substrate may initiate, inhibit or otherwise modulate other reactions or processes producing such detectable signal. As another example, an enzymatic substrate with corresponding reporter enzyme is typically able to generate a detectable signal, e.g., a change in a detectable signal, upon enzymatic reaction.

The gRNA of any of the reaction mixtures, kits, methods, and other embodiments disclosed herein, may have a sequence, e.g., a guide sequence configured to be complementary to any of the target nucleic acid sequences disclosed herein. For example, in embodiments, the target nucleic acid sequence is a sequence from one or more of Sars-COV-2, RSV, FluA, FluB, HIV, and human papilloma virus. In some instances, the CRISPR enzyme comprises trans-cleavage activity, e.g., a Cas12 enzyme or a Cas13 enzyme. In some instances the limit of detection is ˜10 fM. In some instances, the reaction mix does not comprise any reagents for nucleic acid amplification.

In embodiments, a method for detecting a target nucleic acid sequence includes forming a reaction mixture including: a CRISPR system comprising or consisting essentially of: a CRISPR enzyme with nuclease activity and a gRNA, wherein the gRNA is capable of forming a CRISPR system with the CRISPR enzyme and a region of the gRNA is complimentary to a pre-determined target nucleic acid sequence; a complex of a nucleic acid aptamer and a second enzyme; a substrate for the second enzyme; and a biological sample. The method typically includes activating a nuclease activity of the CRISPR enzyme of the CRISPR system by hybridizing (i) a spacer sequence of the gRNA and (ii) a target polynucleotide sequence. The method may include modulating an activity of the second enzyme upon a substrate of the second enzyme by cleaving, with the activated CRISPR enzyme, a nucleic acid aptamer bound to the second enzyme. The reagents of the method, e.g., the CRISPR enzyme, gRNA, second enzyme, e.g., reporter enzyme, and substrate may independently be selected from any of such reagents disclosed herein. The target sequence to which the region of the gRNA is complementary may be any of the target sequences disclosed herein.

In embodiments, the second enzyme is a reporter enzyme, the nucleic acid aptamer is an inhibitor, activator or otherwise modulates the activity of the reporter enzyme, and the biological sample is a biological sample to be assessed for the presence and/or amount of the target nucleic acid sequence. In such embodiments, the action of the reporter enzyme upon its substrate directly or indirectly produces a detectable signal indicative of the presence and/or amount of the target polynucleotide sequence. The method may include detecting the detectable signal and determining the presence and/or amount of the target polynucleotide sequence based on the detected detectable signal. The detectable signal may be any of the detectable signals disclosed herein, e.g., a change in a detectable signal. In any embodiments of the method, the substrate for the reporter enzyme directly or indirectly provides a detectable signal. For example, the substrate for the reporter enzyme may be an enzymatic substrate with corresponding reporter enzyme and is able to generate a change in a detectable signal upon enzymatic reaction.

The biological sample used in the method can be any of the biological samples disclosed herein.

In any embodiments of the method, the nucleic acid aptamer used in the method may be an inhibitor of the second enzyme, e.g., a reporter enzyme, an activator of the second enzyme, e.g., a reporter enzyme, or otherwise a modulator of an activity of the enzyme, e.g., a reporter enzyme.

The nucleic acid aptamer used in the method may have a length of any of the nucleic acid aptamers disclosed herein, e.g., from 20 nucleotides to 100 nucleotides in length.

Performing the method may include cleaving the nucleic acid aptamer, e.g., while the aptamer is in the complex with the reporter enzyme, with a nuclease activity of the CRISPR enzyme.

The nucleic acid aptamer used in any of the methods may be any of the nucleic acid aptamers disclosed herein, e.g., the nucleic acid aptamer may be selected from the group consisting of a single stranded deoxyribonucleic acid (DNA) molecule, a double stranded deoxyribonucleic acid (DNA) molecule, and a ribonucleic acid (RNA) molecule.

The reporter enzyme used in the method may be any the reporter enzymes disclosed herein. For example, the reporter enzyme may be selected from the group consisting of a peroxidase, an oxidase, a phosphatase, a polymerase, or a second CRISPR enzyme. For example, the reporter enzyme may be selected from the group consisting of β-galactosidase, luciferase, alkaline phosphatase, horseradish peroxidase (HRP), β-glucuronidase (GUS). In embodiments, the reporter enzyme is a peroxidase and the aptamer is an activating aptamer for upregulating peroxidase activity. For example, the reporter enzyme peroxidase may be a horseradish peroxidase and the activator aptamer may be a G-quaruplex (G4)-forming DNA aptamer. Such G-quaruplex (G4)-forming DNA aptamer may include or consist of 5′-GGGTGGGTTGGGAGGG-3′ (SEQ ID NO:2). In embodiments, the reporter enzyme is a luciferase and the aptamer is an inhibiting aptamer for inhibiting luciferase activity. For example, the inhibiting luciferase aptamer may include or consist of 5′-ATAGTCTATCGATCAATTGGGTCTGGGTCCGTGCCGGCGTGGTCTTGTAGATAGCAA GTGTATTCA-3′ (SEQ ID NO:3).

Alternatively, e.g., the reporter enzyme used in the method may be a DNA polymerase and the aptamer may be an inhibiting or activating aptamer for the DNA polymerase. In embodiments, the DNA polymerase is a Z05 polymerase and the inhibiting aptamer is a Z05 NTQ aptamer. The Z05 NTQ aptamer may include or consist of SEQ ID NO:1 (5′-CGATCATCTCAGAACATTCTTAGCGTTTTGTTCTTGTGTATGATCG-3′).

In embodiments of the method, the target nucleic acid sequence is a sequence from one or more of Sars-COV-2, RSV, FluA, FluB, HIV, and human papilloma virus.

In embodiments of the method, the CRISPR enzyme comprises trans-cleavage activity. In embodiments of the method, the CRISPR enzyme is a Cas12 enzyme or a Cas13 enzyme.

In embodiments, the method is performed with a limit of detection of the target polynucleotide sequence of no higher than ˜10 fM, e.g., with a limit of detection of ˜10 fM.

In embodiments of the method, the reaction mix does not comprise any reagents for nucleic acid amplification. In embodiments, the method is performed without substantially replicating, e.g., without replicating, or otherwise substantially amplifying, e.g., without amplifying, the target polynucleotide sequence and/or any polynucleotide sequences other than the target polynucleotide sequence.

In any of the reaction mixtures, CRISPR systems, CRISPR enzymes, CRISPR complexes, methods, kits, or other embodiments disclosed herein, the CRISPR enzyme may comprise any CRISPR enzyme having an amino acid sequence having at least 60%, at least 85%, at least 90%, or at least 95% amino acid sequence homogeneity to the amino acid sequence of any of the CRISPR enzymes disclosed in International Application No. PCT/US2024/056904, titled “Systems and Methods of Novel CRISPR-CAS Proteins for In-Vitro Application” and filed 21 Nov. 2024 (“the '904 Application”). For example, such CRISPR enzyme may be one of the CRISPR enzymes disclosed in the '904 Application. Alternatively, or in combination therewith, in any of the reaction mixtures, CRISPR systems, CRISPR enzymes, CRISPR complexes, methods, kits, or other embodiments disclosed herein, the gRNA may be a gRNA comprising a nucleic acid sequence comprising a repeat sequence having at least 60%, at least 85%, at least 90%, or at least 95% nucleic acid homogeneity to the nucleic acid sequence of any of the direct repeat sequences disclosed in the '904 Application. For example, such gRNA may be a gRNA comprising a nucleic acid sequence comprising a repeat sequence disclosed in the '904 Application. As another example, in any of the reaction mixtures, CRISPR systems, CRISPR enzymes, CRISPR complexes, methods, kits, or other embodiments disclosed herein, the CRISPR enzyme may be a CRISPR enzyme having at least 60%, at least 85%, at least 90%, or at least 95% amino acid sequence homogeneity to the amino acid sequence of any of the CRISPR enzymes of any of the CRISPR nucleases (CRISPR enzymes) of the CRISPR nuclease/direct repeat pairs disclosed in the '904 Application. Alternatively, or in combination, the gRNA may be a gRNA comprising a nucleic acid sequence comprising an amino acid sequence having at least 60%, at least 85%, at least 90%, or at least 95% nucleic acid homogeneity to the nucleic acid sequence of any of the direct repeat sequences of any of the CRISPR nuclease/direct repeat pairs disclosed in the '904 Application.

Any of the methods disclosed herein may be performed in part or in whole at a temperature that is about the same as an ambient temperature surrounding the reaction mixture, CRISPR system, CRISPR enzyme, gRNA, aptamer, second enzyme, e.g., reporter enzyme, and/or target polynucleotide. In embodiments, any of the methods disclosed herein are performed in whole or in part without subjecting materials to a heating step. For example, any of the methods disclosed herein performed at an ambient temperature or without a heating step may be performed in part or in whole at a temperature of about 30° C. or less, e.g., at a temperature of 27.5° C. or less or at a temperature of 25° C. or less.

In such embodiments, the methods disclosed herein may include performing, at an ambient temperature, e.g., without subjecting the reagents to a heating step, steps of activating a nuclease activity of the CRISPR enzyme of the CRISPR system by hybridizing (i) a spacer sequence of the gRNA and (ii) a target polynucleotide sequence and/or modulating an activity of a second enzyme, e.g., a reporter enzyme, upon a substrate of the second enzyme by cleaving, with the activated CRISPR enzyme, a nucleic acid aptamer bound to the second enzyme. In any such embodiments, the step of directly or indirectly producing and/or detecting a detectable signal may be performed at an ambient temperature, e.g., without subjecting the reagents to a heating step.

In such embodiments, the CRISPR enzyme may comprise any CRISPR enzyme having an amino acid sequence having at least 60%, at least 85%, at least 90%, or at least 95% amino acid sequence homogeneity to the amino acid sequence of any of the CRISPR enzymes disclosed in the '904 Application, e.g., CRISPR enzyme JW13166 (SEQ ID NO:4). For example, the CRISPR enzyme may comprise or be CRISPR enzyme JW13166. In such embodiments, the gRNA may be or may comprise nucleic acid sequence having at least 85%, at least 90%, or at least 95% homology to SEQ ID NO:5 and/or SEQ ID NO:6.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 (FIG. 1) illustrates a reaction scheme for a reaction mixture comprising a CRISPR system (CRISPR enzyme+gRNA), a complex of an inhibiting nucleic acid aptamer and a reporter enzyme, and a substrate for the reporter enzyme.

FIG. 2A (FIG. 2A) illustrates a reaction scheme for a reaction mixture comprising a CRISPR system (CRISPR enzyme+gRNA) and a signal probe.

FIG. 2B (FIG. 2B) illustrates a reaction scheme for a reaction mixture comprising a reporting enzyme linked to a microbead/surface.

FIG. 2C (FIG. 2C) illustrates a reaction scheme for a reaction mixture comprising an intermediate activator, a secondary activator, and a substrate for the second activator.

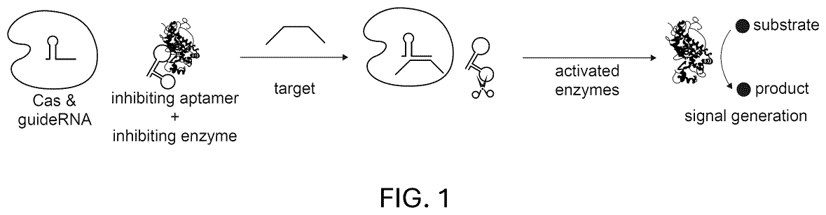

It should be understood that the drawings are not necessarily to scale, and that like reference numbers refer to like features.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Terminology

The singular forms “a,” “an,” and “the” include plural reference unless the context clearly dictates otherwise. Thus, for example, a reference to: “a substrate” includes a plurality of such substrates, so that a substrate “X” (e.g., β-galactosidase, luciferase, alkaline phosphatase, horseradish peroxidase (HRP), β-glucuronidase (GUS)), includes a plurality of substrate molecules X. Similarly, a reference to “a reporter enzyme” includes a plurality of such enzymes, so that a reporter enzyme “Y” (e.g., includes a peroxidase, an oxidase, a phosphatase, a polymerase, or a CRISPR enzyme.” It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The terms “nucleic acid aptamer” or “aptamer” refers to artificially made polynucleotide sequences that target specific reporter enzymes of interest.

The terms “inhibit”, “inhibiting”, and “inhibition” refer to the slowing, halting, or reversing the activity of an enzyme, e.g., a reporter enzyme, or group of such enzymes. The inhibition can be, for example, such that the activity of the enzyme is only about 99% or less, 95% or less, 90% or less, 80% or less, 60% or less, 40%, or less or 20% or less than the enzyme activity that occurs in the absence of the inhibitor under similar or otherwise identical conditions. The terms “activate”, “activating”, and “activation” refer to the increasing, starting, or initiating the activity of an enzyme, e.g., a reporter enzyme, or group of such enzymes. The activation can be, for example, such that the activity of the enzyme is at least about 20%, 40%, 60%, 80%, 90%, 95%, or 99% or greater than the enzyme activity that occurs in the absence of the activator under similar or otherwise identical conditions.

The term specific binding of an aptamer to its target reporter enzyme includes binding with an affinity of at least 10⁻¹, 10⁻², 10⁻³, 10⁻⁴, 10⁻⁵, 10⁻⁶, 10⁻⁷, 10⁻⁸, 10⁻⁹, or 10⁻¹⁰ M⁻¹. Specific binding is generally said to occur when the interaction between the aptamer/enzyme, e.g., reporter enzyme, leads to inhibition, activation, or other modulation of activity by the enzyme. Specific binding can be the result of formation of non-covalent bonds between particular nucleic acids or a particular spatial fit (e.g., lock and key type), or the result of van der Waals forces. Specific binding does not however necessarily imply that the aptamer binds one and only one reporter enzyme.

In the context of formation of a CRISPR complex, “target sequence”, “target polynucleotide sequence”, or “target nucleic acid sequence” refer to a sequence to which a region, e.g., a sequence, e.g., a guide sequence, of a gRNA is designed to have complementarity, where hybridization between a target sequence such region of a gRNA promotes the formation of a CRISPR complex including a CRISPR enzyme, a gRNA including such region, and the target sequence. A target sequence may comprise one or more RNA polynucleotides. The term “target RNA” refers to an RNA polynucleotide being or comprising the target sequence. In other words, the target polynucleotide can be a polynucleotide or a part of a polynucleotide to which a region, e.g., a sequence, e.g., a guide sequence (or a part thereof) of the gRNA is designed to have complementarity with.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form. A polynucleotide is generally composed of a specific sequence of four nucleotide bases: adenine (A); cytosine (C); guanine (G); thymine (T); and uracil (U) for thymine when the polynucleotide is RNA, although in certain microorganism variations of the standard nucleotide bases are known in the art to be present. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “complement” as used herein means the complementary sequence to a nucleic acid according to standard Watson/Crick base pairing rules. A complement sequence can also be a sequence of RNA complementary to the DNA sequence or its complement sequence, and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize fully to a target nucleic acid sequence, positioned 3′ or 5′ to a contiguous sequence of bases. However, substantially complementary sequences still hybridize to a target nucleic acid sequence under moderate or stringent hybridization conditions.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “gene” refers to a polynucleotide containing at least one open reading frame (ORF) that can be transcribed into an RNA (e.g., miRNA, siRNA, mRNA, tRNA, and rRNA) that may encode a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter-none; strand=both; cutoff-60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.goviblast/Blast.cgi. Biologically equivalent polynucleotides are those having the specified percent homology and encoding a polypeptide having the same or similar biological activity.

As used herein, “subject” means a human or non-human and may include, for example, animal strains or species used as “model systems” for research purposes, such a mouse model as described herein. Likewise, subject may include either adults or juveniles (e.g., children). Moreover, subject may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein the abbreviation “CRISPR” generally refers to Clustered Regularly Interspaced Short Palindromic Repeats. Although there are diverse CRISPR-Cas systems among the different species of archaea and bacteria, these systems are generally connected by their dependence on a single-stranded RNA molecule (crRNA), e.g., a gRNA, which guides CRISPR proteins (enzymes) to recognize and cleave nucleic acid targets. The crRNA can be programmed towards a specific target sequence, e.g., a DNA or RNA region of interest, through hybridization to a complementary sequence of the crRNA, e.g., to a guide sequence of a gRNA.

As used herein, the terms, “guide RNA”, “guide nucleic acid”, and “gRNA” refer to a ribonucleic acid sequence capable of forming a CRISPR system with a CRISPR enzyme. The gRNA typically has a conserved sequence forming one or more stem-loop structure(s) (e.g., a direct repeat or “DR”) and a spacer sequence, e.g., a guide sequence. Generally, the CRISPR nuclease (CRISPR enzyme) binds to the gRNA, e.g., to the DR thereof, in the CRISPR system or, in the presence of the target polynucleotide sequence, binds to the gRNA, e.g., to the DR thereof, in the “CRISPR complex” (“complex”). The spacer sequence of the gRNA is a sequence, e.g., a guide sequence, that is complementary to the target nucleic acid and is hybridized therewith in the CRISPR complex.

As used herein the abbreviation “RSV” generally refers to Respiratory Syncytial Virus (RSV). As used herein the abbreviations “FluA” and “FluB” generally refer to Influenza A and Influenza B, the two main types of influenza viruses that cause seasonal flu outbreaks. As used herein the term “Flu” refers to all viruses in the Influenza family. As used herein the abbreviations “SARS-COV-2” refers to the virus causing COVID-19.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” The terms “about” and “approximately” are used interchangeably. Both terms can refer to a variation of +5%, +10%, +20%, or +25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

As used herein, the term “percent sequence identity” with respect to a reference nucleic acid sequence is the percentage of nucleic acid bases in a target sequence that are identical with the nucleic acid bases in the reference sequence, respectively, after aligning the sequences and introducing gaps, if necessary, to achieve the maximum percent sequence identity. Methods of sequence alignment are well known in the art. Optimal alignment of sequences can be conducted by methods described in Needleman and Wunsch, 1970, J. Mol. Biol. 48:443; Pearson and Lipman, 1988, PNAS 85:2444, by computerized implementations of these algorithms.

Alignments can be made using publicly available computer software such as BLASTp, BLASTn, BLAST-2, ALIGN or MegAlign Pro (DNASTAR) software.

As used herein the term “sample”, generally refers to any source of nucleic acids-either from an specimen, from a subject “hosting” the specimen or both—that can be informative of an environment. A sample may refer to a sample derived from a subject, e.g., a biological sample, including or consisting of as a nasal swab, nasopharyngeal swab, sputum, saliva, blood, plasma, serum, urine, tissue, feces, bone marrow, saliva, cerebrospinal fluid, or any other suitable biological, e.g., tissue, sample. A sample may refer to swab samples that are collected from surfaces in food processing facilities, long-term care facilities, hospitals, restaurants, or any suitable surface comprising nucleic acids. A sample may be a sample that comprises a biological tissue, soil, water, air, air filter materials, animal production, feed, manure, crop production, manufacturing plants, or any other suitable samples. Such samples may be, e.g., derived from a hospital or a clinic and they may be analyzed on a mobile platform.

“Detecting” as used herein refers to determining the presence and/or amount, e.g., degree, of a target nucleic acid sequence from a sample. Detection does not require the method to provide 100% sensitivity and/or 100% specificity.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

Statistically significant means p≤0.05.

### DETAILED DESCRIPTIONS

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

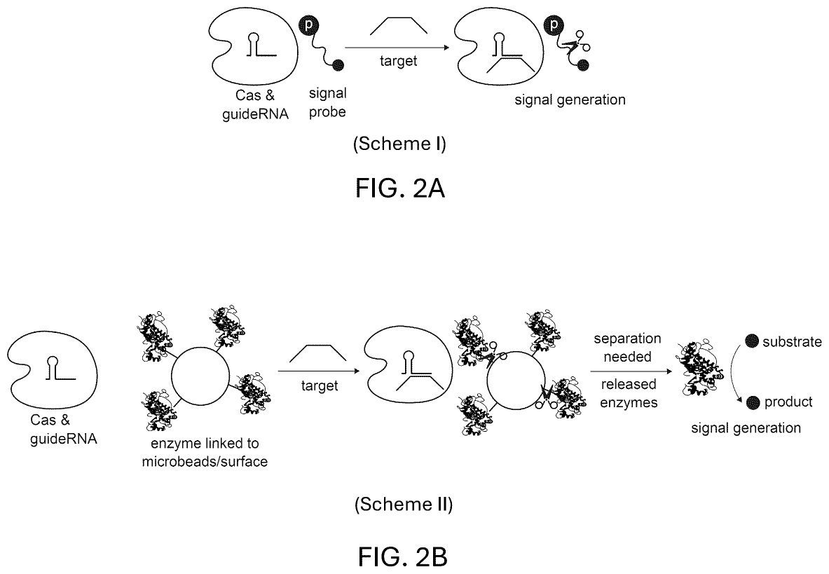

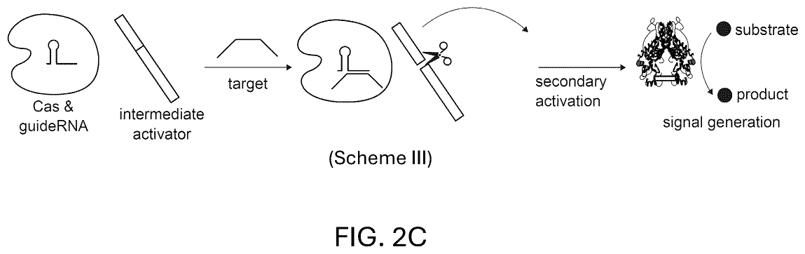

Several CRISPR-Cas based diagnostics described in the art rely on the principle that, once activated, certain CRISPR-Cas enzyme(s) can cleave labelled nucleic acid materials and release molecules that generate a detectable signal, e.g. fluorophore, electrochemical probe, and other suitable signal generating molecules). See, e.g., Kellner, M. J., Koob, J. G., Gootenberg, J. S. et al. SHERLOCK: nucleic acid detection with CRISPR nucleases. Nat Protoc 14, 2986-3012 (2019); Cascade CRISPR/cas enables amplification-free microRNA sensing with fM-sensitivity and single-base-specificity; see also Yong Sha, Ru Huang, Mengqi Huang, Huahua Yue, Yuanyue Shan, Jiaming Hu and Da Xing; Chem. Commun., 2021, 57, 247-250; see further Nucleic acid detection with CRISPR-Cas13a/C2c2, Jonathan S. Gootenberg, Omar O. Abudayyeh, Jeong Wook Lee, Patrick Essletzbichler, Aaron J. Dy, Julia Joung, Vanessa Verdine, Nina Donghia, Nichole M. Daringer, Catherine A. Freije, Cameron Myhrvold, Roby P. Bhattacharyya, Jonathan Livny, Aviv Regev, Eugene V. Koonin, Deborah T. Hung, Pardis C. Sabeti, James J. Collins, Feng Zhang, Science. 2017 Apr. 28; 356 (6336): 438-442. Illustrative examples of this principle are shown in FIG. 2A, scheme I. However, one disadvantage of the reaction illustrated by scheme I is that assay sensitivity is often weak. This is because the reaction rate and sensitivity is limited by the Cas activity and there is no further amplification of the signal, either by nucleic acid amplification or another means. To address such limitations, the art generally teaches that PCR amplification methods should be introduced to increase the signal. See, Liu, T. Y., Knott, G. J., Smock, D. C. J. et al. Accelerated RNA detection using tandem CRISPR nucleases. Nat Chem Biol 17, 982-988 (2021), see also Target-triggered CRISPR-Cas13a autocatalysis-driven amplification strategy for one-step detection of circadian clock gene Zhiyuan Feng, Yi Xue, Yangfang Yun, Zheng Liu and Jingjing Zhang, Sens. Diagn., 2023, 2, 632-639.

To attempt to address such challenges, some in the art developed heterogeneous reaction schemes, such as the reaction scheme shown in FIG. 2B, scheme II. See, e.g., Enhancing CRISPR-Cas-Mediated Detection of Nucleic Acid and Non-nucleic Acid Targets Using Enzyme-Labeled Reporters. Devleena Samanta, Sasha B. Ebrahimi, Namrata Ramani, and Chad A. Mirkin. J. Am. Chem. Soc. 2022, 144, 36, 16310-16315. As illustrated in the diagram of FIG. 2B, the signal enzyme is pre-modified to be linked to a surface such as microbeads via nucleic acid linker. When the target is present, a CRISPR-Cas enzyme system is activated and it cleaves a nucleic acid linker thereby releasing the CRISPR-Cas enzyme. After a separation step, these released enzymes can provide a readout based on their corresponding reactions. However, one significant disadvantage of this scheme II is the requirement for a separation step. See, e.g., Enhancing CRISPR-Cas-Mediated Detection of Nucleic Acid and Non-nucleic Acid Targets Using Enzyme-Labeled Reporters, Devleena Samanta, Sasha B. Ebrahimi, Namrata Ramani, and Chad A. Mirkin, J. Am. Chem. Soc. 2022, 144, 36, 16310-16315 (abstract illustrates steps for removal of beads). The need to separate released and unreleased enzymes adds additional steps to the workflow and overall liquid handling, which in turn limit the use and application of the method.

Yet another method developed in the art is a heterogenous reaction illustrated in FIG. 2C, scheme III. As illustrated in the diagram of FIG. 2C, an activated CRISPR-Cas enzyme cleaves a substrate and generates an intermediate activator, which then activates a secondary signal enzyme without the need for separation steps. However, the disadvantage of the scheme shown in FIG. 2C is that necessarily imposes very specific requirements regarding the design of the intermediate activator and the choice of secondary enzymes. As a results, there are very limited number of CRISPR systems that can achieve scheme III, because the Cas reaction directly links to secondary enzyme activator.

The present disclosure provides novel reaction mixtures, kits, systems and methods that are generally applicable to a wide range of CRISPR enzymes and typically do not require any separation steps. The disclosure provides mixtures, kits, systems and methods that add another level of CRISPR based enzymatic reactions to generate a more detectable detectable signal. Briefly, the present disclosure provides reaction mixtures, kits, systems and methods comprising or using: a CRISPR-Cas enzyme (CRISPR enzyme), a gRNA, an aptamer, and a second enzyme, e.g., a reporter enzyme (e.g., a signaling enzyme). The CRISPR enzyme and gRNA may be in a CRISPR system comprising, consisting essentially of, or consisting of: the CRISPR enzyme and the gRNA or the CRISPR enzyme and gRNA may be initially separate and then form such CRISPR system in the reaction mixture. The gRNA includes a region, e.g., a guide sequence, that is complimentary to a pre-determined target nucleic acid sequence

The aptamer is configured to complex with and modulate the activity of the second enzyme, e.g., by inhibition, activation, or other modulation. For example, while in such complex, an inhibiting aptamer binds to the second enzyme resulting in inhibited enzyme activity. When a target nucleic acid is present in the reaction, the CRISPR-Cas/gRNA system becomes activated and collaterally cleaves nucleic acids in the solution, including the aptamer bound to the second enzyme. Once the aptamer gets cleaved, the second enzyme is no longer be inhibited, activated, or otherwise modulated by the aptamer. If, for example, the aptamer is an inhibitor of the second enzyme, such cleavage results in an increase of second enzyme activity. This additional enzyme activity can directly or indirectly generate an increased detectable signal if corresponding substrate exists in the system. As another example, if the aptamer is an activator of the second enzyme, such cleavage results in a decrease of second enzyme activity. This reduced activity can directly or indirectly generate a decreased detectable signal if the corresponding substrate exists in the system.

In some aspects, the disclosure relates to enzymatic cascade reactions for achieving better sensitivity in detecting a target nucleic acid sequence. Several aspects of the disclosure relate to a reaction mixture comprising: a CRISPR enzyme; a gRNA, wherein the gRNA is in a CRISPR system with the CRISPR enzyme or is configured to form a CRISPR system with the CRISPR enzyme, and a region of the gRNA is complimentary to a pre-determined target nucleic acid sequence; an aptamer (e.g., an inhibiting aptamer or an activating aptamer); a second enzyme, e.g., a reporter enzyme (“signaling” enzyme); and, optionally, a substrate for the second enzyme, e.g., a substrate for the reporter enzyme. The aptamer interacts with the second enzyme and forms a complex, resulting in, e.g., inhibited signaling enzyme activity or “bolstered” (activated) enzyme activity. When a target is present in the reaction mix, the Cas/guideRNA system becomes activated and preferentially collaterally cleave(s) all nucleic acids in the solution, including the aptamer. Once the aptamer gets cleaved, the inhibitory, activating, or other modulating effect of the aptamer on the second enzyme is decreased in magnitude or eliminated. Where the aptamer is an inhibitory aptamer, the second enzyme becomes free to act upon its substrate, e.g., when the second enzyme is a reporter enzyme, the reporter enzyme becomes free to directly or indirectly produce or increase a detectable signal. Where the aptamer is an activating aptamer, the second enzyme decreases its rate of activity toward the substrate, e.g., when the second enzyme is a reporter enzyme, the reporter enzyme directly or indirectly ceases or decreases a rate of production of a detectable signal. In the presence of a substrate, such reporter (signaling) enzyme activity produces a robust dectectable signal or change in detectable signal that significantly improves the limit-of-detection of other techniques in the art.

It is contemplated that cascade enzymatic reaction systems of the disclosure achieve better sensitivity compared to conventional Cas reactions. It is further contemplated that such systems and approaches could be widely applicable to different aptamer/enzyme systems, making it a versatile approach to couple with a variety of enzymatic biosensors. Further yet, such cascade reactions can be incorporated in a homogeneous solution without any needed separation step, resulting in a simplified workflow.

### Nucleic Acid Aptamers, Reporter Enzymes, and Reporter Enzyme Substrates

Generally, due to their ability to fold into distinct three-dimensional structures, single-stranded nucleic acids can bind target molecules with high specificity and affinity. In reference to the Latin word aptus meaning “to fit”, these nucleic acid receptors have been named “aptamers.” A repertoire of DNA and RNA aptamers that can interact with a diverse assortment of ligands ranging from ions, small molecules, peptides, single proteins, organelles, viruses, and even whole cells have been reported in the literature, as well as the methodologies for identifying such aptamers. See, e.g., Wilson D. S., Szostak J. W. In vitro selection of functional nucleic acids. Annu. Rev. Biochem. 1999; 68:611-647. The disclosure contemplates aptamers developed with such methods that can either inhibit or modulate the activity of CRISPR enzymes, common reporter, e.g., biosensing, enzymes (such as oxidases, peroxidases, phosphatases), and/or polymerases.

In vitro selection processes, such as SELEX (Systematic Evolution of Ligands by EXponential enrichment), are used to identify aptamers that specifically bind to a reporter enzyme. During SELEX, a random pool of nucleic acids is subjected to iterative rounds of selection, amplification, and enrichment until high-affinity aptamers for the contemplated CRISPR enzymes, reporter enzymes, and/or polymerases are obtained.

In certain instances, a reaction mixture of the disclosure comprises a complex of a nucleic acid aptamer and a reporter enzyme, in addition to a substrate for the reporter enzyme. A plurality of reporter enzymes are contemplated by the disclosure, and each is capable of directly or indirectly facilitating generation of a detectable signal, such as a colorimetric, chemiluminescent, or fluorescent signal, that is inhibited, activated or otherwise modulated such that the enzyme's activity is altered by the binding of one or more nucleic acid aptamers (e.g., DNA or RNA) to the enzyme. Upon activation of the CRISPR systems disclosed herein, the one or more nucleic acid aptamers (e.g., DNA or RNA) are cleaved or degraded to an extent that they no longer inhibit, activate, or otherwise modulate the enzyme's ability to generate the detectable signal. In certain example embodiments, the aptamer is an inhibiting aptamer of Roche's Z05 polymerase enzyme. In certain example embodiments the inhibiting aptamer has a sequence of Z05 NTQ aptamer X (SEQ ID NO:1: 5′-CGATCATCTCAGAACATTCTTAGCGTTTTGTTCTTGTGTATGATCG-3′). When this inhibiting aptamer is cleaved, Roche's Z05 polymerase enzyme will become active and will amplify a signal via polymerization of one or more target nucleic acid sequences in the reaction mix.

It is contemplated in several embodiments that the reporter enzyme is a DNA polymerase. Non-limiting examples of DNA polymerases includes, for instance, Anaerocellum thermophilum DNA polymerase, Bacillus pallidus DNA polymerase, Bacillus stearothermophilus DNA polymerase, Carboxydothermus hydrogenoformans DNA polymerase, Thermoactinomyces vulgaris DNA polymerase, Thermoanaerobacter thermohydrosulfuricus DNA polymerase, Thermosipho africanus DNA polymerase, Thermotoga neapolitana DNA polymerase, Thermus aquaticus DNA polymerase, Thermus thermophilus DNA polymerase, or Thermus Z05 DNA polymerase. The invention contemplates reaction mixtures with aptamers that are inhibitors of the one or more aforementioned enzymes. In particular instances, the aptamer binds to the nuclease at low temperatures and does not bind at elevated temperatures.

In any embodiment where the reporter enzyme is a DNA polymerase, the reaction mixture may further contain a reverse transcriptase, primers, and other reagents suitable to facilitate action of such DNA polymerase.

In certain example embodiments, the aptamer is an inhibiting aptamer of a luciferase enzyme. In certain example embodiments the luciferase inhibiting aptamer has a sequence of 5′-ATAGTCTATCGATCAATTGGGTCTGGGTCCGTGCCGGCGTGGTCTTGTAGATAGCAA GTGTATTCA-3′ (SEQ ID NO:3). When this aptamer is cleaved, luciferase enzyme will cleave a peptide colorimetric or fluorescent substrate. In certain example embodiments, the colorimetric substrate is para-nitroanilide (pNA) covalently linked to the peptide substrate foluciferase. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. Inhibiting aptamers may also be used for horseradish peroxidase (HRP), beta-galactosidase, or calf alkaline phosphatase (CAP) and within the general principals laid out above.

In certain embodiments, activity of a CRISPR system (either DNAase or RNAse activity) is detected colorimetrically via cleavage of enzyme-activating aptamers. One potential mode of converting DNAse or RNAse activity into a colorimetric signal is to couple the cleavage of a DNA or RNA aptamer with the de-activation of an enzyme that is capable of producing a colorimetric output. In the absence of RNA or DNA cleavage, the intact aptamer will bind to the enzyme target and stimulate its activity. The advantage of this readout system is that the enzyme provides an additional amplification step: once liberated from an aptamer via collateral activity (e.g. Cpf1 collateral activity), the colorimetric enzyme will continue to produce colorimetric product, leading to a multiplication of signal.

In certain embodiments, an aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In certain embodiments, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In certain embodiments, the nucleic acid aptamer may be a DNA or RNA aptamer and/or may comprise a detectable ligand and/or a masking component are attached.

In preferred instances, the nucleic acid aptamer may sequester a reporter enzyme, wherein the reporter enzyme directly or indirectly generates a detectable signal upon release from the aptamer by acting upon a substrate. In some embodiments, the nucleic acid aptamer may be an inhibitor aptamer that inhibits an enzyme and prevents the enzyme from catalyzing generation of a detectable signal from a substance. In some embodiments, the nucleic acid aptamer may form a tertiary complex that activates the activity of an enzyme and may catalyze generation of a detectable signal from a substrate.

In certain embodiments, detection of a target nucleic acid in a reaction mix is achieved by activation of a CRISPR system that is detected colorimetrically via formation and/or activation of G-quadruplexes. G quadruplexes in DNA can complex with heme (iron (III)-protoporphyrin IX) to form a DNAzyme with peroxidase activity. When supplied with a peroxidase substrate (e.g., ABTS: (2,2′-Azinobis [3-ethylbenzothiazoline-6-sulfonic acid]-diammonium salt)), the G-quadruplex-heme complex in the presence of hydrogen peroxide causes oxidation of the substrate, which then forms a green color in solution. An example G-quadruplex forming DNA sequence is: GGGTGGGTTGGGAGGG (SEQ ID NO:2). By hybridizing an additional DNA or RNA sequence, referred to herein as a “staple,” to this DNA aptamer, formation of the G-quadraplex structure will be limited. Upon collateral activation, the staple will be cleaved allowing the G quadraplex to form and heme to bind. This strategy is particularly appealing because color formation is enzymatic, meaning there is additional amplification beyond collateral activation.

In certain embodiments, the substrate for the reporter enzyme may comprise an RNA oligonucleotide designed to bind a G-quadruplex forming sequence, wherein a G-quadruplex structure is formed by the G-quadruplex forming sequence upon cleavage of the nucleic acid aptamer, and wherein the G-quadruplex structure generates a detectable positive signal.

In certain example embodiments, the substrate for the reporter enzyme may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the detection construct are able to generate a detectable signal, for example by aggregation or simple increase in solution concentration. In certain example embodiments, the immobilized nucleic acid aptamer that can be cleaved by the activated CRISPR system upon detection of a target molecule.

In one example embodiment, the substrate for the reporter enzyme comprises an agent that changes color depending on whether the agent is aggregated or dispersed in solution. For example, certain nanoparticles, such as colloidal gold, undergo a visible purple to red color shift as they move from aggregates to dispersed particles. Accordingly, in certain example embodiments, such agents may be held in aggregate by one or more bridge molecules. At least a portion of the bridge molecule may comprises RNA or DNA. Upon activation of the CRISPR systems disclosed herein, the RNA or DNA portion of the bridge molecule is cleaved allowing the detection agent to disperse and resulting in the corresponding change in color. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

In some instances, the disclosure contemplates aptamer/reporting enzyme complexes with a pre-determined dissociation constant of the aptamer for its target reporting enzyme. An affinity between a nucleic acid aptamer and a reporter enzyme can be selected during aptamer development to be an affinity of at least 10⁻¹, 10⁻², 10⁻³, 10⁻⁴, 10⁻⁵, 10⁻⁶, 10⁻⁷, 10⁻⁸, 10⁻⁹, or 10⁻¹⁰ M⁻¹.

The size of nucleic acid aptamers can vary widely, but they are typically in the range of 20 to 100 nucleotides. The size of an aptamer is influenced by several factors, including the target molecule itself. In some aspects the disclosure contemplates nucleic acid aptamers that are 20 nucleotides to 100 nucleotides in length. In some aspects, the disclosure contemplates aptamers that are 10 nucleotides to 150 nucleotides, 10 nucleotides to 140 nucleotides, 10 nucleotides to 130 nucleotides, 10 nucleotides to 120 nucleotides, 10 nucleotides to 110 nucleotides, 10 nucleotides to 100 nucleotides, 10 nucleotides to 90 nucleotides, 10 nucleotides to 80 nucleotides, 10 nucleotides to 70 nucleotides, 10 nucleotides to 60 nucleotides, 10 nucleotides to 50 nucleotides, 10 nucleotides to 40 nucleotides, 10 nucleotides to 30 nucleotides, 20 nucleotides to 150 nucleotides, 20 nucleotides to 140 nucleotides, 20 nucleotides to 130 nucleotides, 20 nucleotides to 120 nucleotides, 20 nucleotides to 110 nucleotides, 20 nucleotides to 100 nucleotides, 20 nucleotides to 90 nucleotides, 20 nucleotides to 80 nucleotides, 20 nucleotides to 70 nucleotides, 20 nucleotides to 60 nucleotides, 20 nucleotides to 50 nucleotides, 20 nucleotides to 40 nucleotides, and or 20 nucleotides to 30 nucleotides in length. In some aspects, the disclosure contemplates aptamers that are 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, 80 nucleotides, 81 nucleotides, 82 nucleotides, 83 nucleotides, 84 nucleotides, 85 nucleotides, 86 nucleotides, 87 nucleotides, 88 nucleotides, 89 nucleotides, 90 nucleotides, 91 nucleotides, 92 nucleotides, 93 nucleotides, 94 nucleotides, 95 nucleotides, nucleotides, 96 nucleotides, 97 nucleotides, 98 nucleotides, 99 nucleotides, 100 nucleotides, 101 nucleotides, 102 nucleotides, 103 nucleotides, 104 nucleotides, 105 nucleotides, 106 nucleotides, 107 nucleotides, 108 nucleotides, 109 nucleotides, 110 nucleotides, 111 nucleotides, 112 nucleotides, 113 nucleotides, 114 nucleotides, 115 nucleotides, 116 nucleotides, 117 nucleotides, 118 nucleotides, 119 nucleotides, 120 nucleotides, 121 nucleotides, 122 nucleotides, 123 nucleotides, 124 nucleotides, 125 nucleotides, 126 nucleotides, 127 nucleotides, 128 nucleotides, 129 nucleotides, 130 nucleotides, 131 nucleotides, 132 nucleotides, 133 nucleotides, 134 nucleotides, 135 nucleotides, 136 nucleotides, 137 nucleotides, 138 nucleotides, 139 nucleotides, 140 nucleotides, 141 nucleotides, 142 nucleotides, 143 nucleotides, 144 nucleotides, 145 nucleotides, 146 nucleotides, 147 nucleotides, 148 nucleotides, 149 nucleotides, and/or 150 nucleotides long.

In certain instances, the disclosure contemplates nucleic acid aptamers that are a single stranded deoxyribonucleic acid (DNA) molecule and cleavable by certain CRISPR-Cas systems with collateral cleavage activity, e.g., Cas12 and others. In certain instances, the disclosure contemplates nucleic acid aptamers that are a double stranded deoxyribonucleic acid (DNA) molecule, and cleavable by certain CRISPR-Cas systems with collateral cleavage activity, e.g., Cas12 and others.

In certain instances, the disclosure contemplates nucleic acid aptamers that are a ribonucleic acid (RNA) molecule and cleavable by certain CRISPR-Cas systems with collateral cleavage activity, e.g. Cas13 and others.

A reporter enzyme can be any enzyme capable of directly or indirectly producing a readable signal. In some cases, a reporter enzyme is selected from the group consisting of a peroxidase, an oxidase, a phosphatase, a polymerase, or a CRISPR enzyme. Specifically, in certain cases, the reporter enzyme is selected from the group consisting of β-galactosidase, luciferase, alkaline phosphatase, horseradish peroxidase (HRP), β-glucuronidase (GUS). Non-limiting exemplary aptamers considered by the disclosure for complexing with the aforementioned reporter enzymes are listed in Table 1.

TABLE 1

| | Target | |
|---|---|---|
| | Reporting | Type of |
| Aptamer Sequence | Enzyme | Interaction |
| Z05 NTQ aptamer | Roche Z05 | Inhibitory |
| 5′-CGATCATCTCAGAACATTCTTAG | polymerase | |
| CGTTTTGTTCTTGTGTATGATCG-3′ | | |
| (SEQ ID NO: 1) | | |
| 5′-GGGTGGGTTGGGAGGG-3′ | Peroxidase | Modulatory |
| (SEQ ID NO: 2) | Luciferase | Inhibitory |
| 5′-ATAGTCTATCGATCAATTGGGTC | | |
| TGGGTCCGTGCCGGCGTGGTCTTGTA | | |
| GATAGCAAGTGTATTCA-3′ | | |
| (SEQ ID NO: 3) | | |

Furthermore, the aptamer discovery process readily permits modification, Such as aptamer sequence optimization and the minimization of aptamer length. See, e.g., Conrad et al. 1996: Eaton et al. 1997: Cload et al., The Aptamer Hand book, 363-416 (2006); and Wilson et al., Curr Opin Chem Biol, 10 (6), 607-614 (2006). Additionally, 2′-modifications, such as 2′-fluoro and 2′-O-Me, may be utilized for stabilization against nucleases without compromising the aptamer binding interaction with the target. See, e.g., Lin et al., Nucleic Acids Res., 22, 5229-5234 (1994); Jellinek et al., Biochemistry, 34, 11363-1137 (1995); Lin et al., Nucleic Acids Res., 22, 5229-5234 (1994); Kubik et al., J. Immunol., 159 (1), 259-267 (1997); Pagratis et al., Nat. Biotechnol., 1, 68-73 (1997); and Wilson et al., Curr Opin Chem Biol, 10 (6), 607-614 (2006).

Chemical substitutions have been incorporated into libraries of transcripts from which aptamers are discovered with the view towards selecting aptamers with various characteristics, such as increased target affinity. See, e.g., Latham et al., Nucleic Acids Res., 22, 2817-2822 (1994); Vaish et al., Biochemistry, 42, 8842-8851 (2003); Saitoh et al., Nucleic Acids Res. Suppl., 2, 215-216 (2002); Masud et al., Bioorg. Med. Chem., 12, 1111-1120 (2004); King et al., Biochemistry, 41,9696-9706 (2002); and Yang, X. and Gorenstein, D. G., Curr. Drug Targets, 5, 705-715 (2004). In some instances, the present disclosure contemplates aptamers with such chemical modifications.

One of ordinary skill in the art will understand that certain aptamers described herein are analyzed using standard techniques in the field, which include liquid chromatography and mass spectrometry, such as electron spray ionization liquid chromatography mass spectrometry, polyacrylamide gel electrophoresis or capillary electrophoresis to evaluate aptamer reporter enzyme complexes. In some embodiments, the analyzing the aptamer on its own or analyzing the aptamer/reporter enzyme complex comprises analyzing the resulting aptamer using a bioanalytical method selected from the group consisting of one or more of denaturing polyacrylamide gel electrophoresis (PAGE), capillary electrophoresis; HPLC and LC/MS, particularly LC/MS/MS or LC/MS/MS/MS, and more particularly ESI LC/MS, ESI-LC/MS/MS and ESI-LC/MS/MS/MS.

An aptamer as described herein can be synthesized using any oligonucleotide synthesis techniques known in the art including solid phase oligonucleotide synthesis techniques (see, e.g., Froehler et al., Nucl. Acid Res., 14:5399-5467 (1986) and Froehler et al., Tet. Lett., 27:5575-5578 (1986)) and solution phase methods, such as triester synthesis methods (see, e.g., Sood et al., Nucl. Acid Res., 4:2557 (1977) and Hirose et al., Tet. Lett., 28:2449 (1978)).

The substrate for the reporter enzyme can be present in the reaction mixture in any suitable concentration for detection by the reporter enzyme. In some cases, the substrate is present in the reaction mix in a concentration ranging from 10 nM to 1,000 nM, from 20 nM to 1,000 nM, from 30 nM to 1,000 nM, from 40 nM to 1,000 nM, from 50 nM to 1,000 nM, from 60 nM to 1,000 nM, from 70 nM to 1,000 nM, from 80 nM to 1,000 nM, from 90 nM to 1,000 nM, from 100 nM to 1,000 nM, from 110 nM to 1,000 nM, from 120 nM to 1,000 nM, from 130 nM to 1,000 nM, from 140 nM to 1,000 nM, from 150 nM to 1,000 nM, from 160 nM to 1,000 nM, from 170 nM to 1,000 nM, from 180 nM to 1,000 nM, from 190 nM to 1,000 nM, from 200 nM to 1,000 nM, from 210 nM to 1,000 nM, from 220 nM to 1,000 nM, from 230 nM to 1,000 nM, from 240 nM to 1,000 nM, from 250 nM to 1,000 nM, 10 nM to 750 nM, from 20 nM to 750 nM, from 30 nM to 750 nM, from 40 nM to 750 nM, from 50 nM to 750 nM, from 60 nM to 750 nM, from 70 nM to 750 nM, from 80 nM to 750 nM, from 90 nM to 750 nM, from 100 nM to 750 nM, from 110 nM to 750 nM, from 120 nM to 750 nM, from 130 nM to 750 nM, from 140 nM to 750 nM, from 150 nM to 750 nM, from 160 nM to 750 nM, from 170 nM to 750 nM, from 180 nM to 750 nM, from 190 nM to 750 nM, from 200 nM to 750 nM, from 210 nM to 750 nM, from 220 nM to 750 nM, from 230 nM to 750 nM, from 240 nM to 750 nM, from 250 nM to 750 nM, 10 nM to 500 nM, from 20 nM to 500 nM, from 30 nM to 500 nM, from 40 nM to 500 nM, from 50 nM to 500 nM, from 60 nM to 500 nM, from 70 nM to 500 nM, from 80 nM to 500 nM, from 90 nM to 500 nM, from 100 nM to 500 nM, from 110 nM to 500 nM, from 120 nM to 500 nM, from 130 nM to 500 nM, from 140 nM to 500 nM, from 150 nM to 500 nM, from 160 nM to 500 nM, from 170 nM to 500 nM, from 180 nM to 500 nM, from 190 nM to 500 nM, from 200 nM to 500 nM, from 210 nM to 500 nM, from 220 nM to 500 nM, from 230 nM to 500 nM, from 240 nM to 500 nM, from 250 nM to 500 nM, 10 nM to 450 nM, from 20 nM to 450 nM, from 30 nM to 450 nM, from 40 nM to 450 nM, from 50 nM to 450 nM, from 60 nM to 450 nM, from 70 nM to 450 nM, from 80 nM to 450 nM, from 90 nM to 450 nM, from 100 nM to 450 nM, from 110 nM to 450 nM, from 120 nM to 450 nM, from 130 nM to 450 nM, from 140 nM to 450 nM, from 150 nM to 450 nM, from 160 nM to 450 nM, from 170 nM to 450 nM, from 180 nM to 450 nM, from 190 nM to 450 nM, from 200 nM to 450 nM, from 210 nM to 450 nM, from 220 nM to 450 nM, from 230 nM to 450 nM, from 240 nM to 450 nM, from 250 nM to 450 nM, 10 nM to 400 nM, from 20 nM to 400 nM, from 30 nM to 400 nM, from 40 nM to 400 nM, from 50 nM to 400 nM, from 60 nM to 450 nM, from 70 nM to 450 nM, from 80 nM to 450 nM, from 90 nM to 450 nM, from 100 nM to 400 nM, from 110 nM to 400 nM, from 120 nM to 400 nM, from 130 nM to 400 nM, from 140 nM to 400 nM, from 150 nM to 400 nM, from 160 nM to 400 nM, from 170 nM to 400 nM, from 180 nM to 400 nM, from 190 nM to 400 nM, from 200 nM to 400 nM, from 210 nM to 400 nM, from 220 nM to 400 nM, from 230 nM to 400 nM, from 240 nM to 400 nM, from 250 nM to 400 nM.

In some cases, the substrate is present in the reaction mix in a concentration of no more than 10 nM, no more than 20 nM, no more than 30 nM, no more than 40 nM, no more than 50 nM, no more than 60 nM, no more than 70 nM, no more than 80 nM, no more than 90 nM, no more than 100 nM, no more than 110 nM, no more than 120 nM, no more than 130 nM, no more than 140 nM, no more than 150 nM, no more than 160 nM, no more than 170 nM, no more than 180 nM, no more than 190 nM, no more than 200 nM, no more than 210 nM, no more than 220 nM, no more than 230 nM, no more than 240 nM, no more than 250 nM, no more than 260 nM, no more than 270 nM, no more than 280 nM, no more than 290 nM, no more than 300 nM, no more than 310 nM, no more than 320 nM, no more than 330 nM, no more than 340 nM, no more than 350 nM, no more than 360 nM, no more than 370 nM, no more than 380 nM, no more than 390 nM, no more than 410 nM, no more than 420 nM, no more than 430 nM, no more than 440 nM, no more than 450 nM, no more than 460 nM, no more than 470 nM, no more than 480 nM, no more than 490 nM, no more than 500 nM, no more than 510 nM, no more than 520 nM, no more than 530 nM, no more than 540 nM, no more than 550 nM, no more than 560 nM, no more than 570 nM, no more than 580 nM, no more than 590 nM, no more than 600 nM, no more than 610 nM, no more than 620 nM, no more than 630 nM, no more than 640 nM, no more than 650 nM, no more than 660 nM, no more than 670 nM, no more than 680 nM, no more than 690 nM, no mora than 700 nM, no more than 710 nM, no more than 720 nM, no more than 730 nM, no more than 740 nM, no more than 750 nM, no more than 800 nM, no more than 850 nM, no more than 900 nM, no more than 950 nM, no more than 1,000 nM, no more than 2,000 nM, no more than 3,000 nM, no more than 4,000 nM, no more than 5,000 nM, no more than 6,000 nM, no more than 7,000 nM, no more than 8,000 nM, or another suitable amount.

### CRISPR Systems

Since their initial discovery, the number of different CRISPR-Cas systems has expanded rapidly. Currently, CRISPR-Cas systems can be divided, according to evolutionary relationships, into two classes, six types and several subtype. The classes of CRISPR-Cas system are defined by the nature of the ribonucleoprotein effector complex: class 1 systems are characterized by a complex of multiple effector proteins, and class 2 systems encompass a single crRNA-binding protein. The main difference between CRISPR type II (Cas9) systems and type V (Cas12) and type VI (Cas13) systems is the ability of the latter two systems to trigger non-specific collateral cleavage (trans cleavage) on target recognition. Collateral activity involves the cleavage of non-targeted single-stranded DNA (ssDNA; Cas12) or single-stranded RNA (ssRNA; Cas13) in solution, which enables the sensing of nucleic acids through signal amplification and allows for various readouts through the addition of functionalized reporter nucleic acids, which are generally cleaved by collateral activity.

In some configurations, the disclosure contemplates reaction mixtures comprising one or more, and/or two or more, CRISPR-systems within the same reaction mixture (enzymes, gRNAs, substrates, co-factors) with trans-cleavage activity. In some configurations, one CRISPR system is applied as a reporter enzyme, while another CRISPR system is applied for the detection of a target nucleic acid. Upon activation by a target nucleic acid the CRISPR enzymes with trans-cleavage activity, (e.g., Cas12 and Cas13) indiscriminately cleave short single-stranded reporter oligos. The art teaches embodiments where target nucleotides are labeled with reporter molecules, and cleavage of such targets separates the fluorophore from the quencher which are typically labeled at opposite ends of the reporter molecule, and generating measurable fluorescence signals. Such signals, however, are limited by the ability of the CRISPR systems to cleave the labeled target nucleic acid. The present disclosure contemplates alternative embodiments where activation of a CRISPR system by a target nucleic acid leaves to further activation of a second CRISPR system by the cleaved target nucleic acid; therefore amplifying the detected signal in signaling cascade. Collateral activity of class 2 CRISPR-Cas systems includes Cas13b and Cas 12a enzymes, as described in Gootenberg et al. Science, 2018 Apr. 27; 360 (6387): 439-444, incorporated herein by reference. In certain example embodiments, a Cas that has collateral activity (e.g., collateral nucleic acid cleavage activity) that can be included in the CRISPR-Cas system is a Cas13 (e.g. a Cas13a, 13b, Cas13c and/or Cas13d). In certain example embodiments, a Cas that has collateral activity that can be included in the CRISPR-Cas system is a Cas12 (e.g., Cas 12a, 12b, 12c, 12cl, 12c2, 12d, 12e, 12al, 12gl, 12hl, 12il, 12f (also known as Cas14)).

In certain example embodiments, the CRISPR-Cas system includes a Cas13 (e.g. a Cas13a, 13b, Cas13c and/or Cas13d). In particular embodiments, the Type VI RNA-targeting Cas enzyme is C2c2. In other example embodiments, the Type VI RNA-targeting Cas enzyme is Cas 13b. In particular embodiments, the homologue or orthologue of a Type VI protein such as C2c2 as referred to herein has a sequence homology or identity of at least 30%, or at least 40%, or at least 50%, or at least 60%, or at least 70%, or at least 80%, more preferably at least 85%, even more preferably at least 90%, such as for instance at least 95% with a Type VI protein such as C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2). In further embodiments, the homologue or orthologue of a Type VI protein such as C2c2 as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnob acterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2).

In certain example embodiments, a Cas that has collateral activity that can be included in the CRISPR-Cas system is or includes one or more Cas 12 polypeptides (e.g., Cas 12a (also known as Cpf1), 12b (also known as C2c1), 12c, 12c1, 12c2, 12d, 12e, 12a1, 12g1, 12h1, 12il, 12f (also known as Cas14) See e.g., Kaminski et al., Nat. Biomed. Eng. 5:643-656 (2021)). In some embodiments, the Cas12 protein can have trans-cleavage activity (also referred to as collateral cleavage), which cleaves ssDNA indiscriminately. In some embodiments, the Cas 12 has multiple-turnover nuclease activity, which can be harnessed in the context of an assay described herein for amplified detection of targets.

In CRISPR-based diagnostics, quantification through comparison with a standard curve can be achieved within the picomolar-to-micromolar range (10⁻¹² M-10⁻⁶ M), where CRISPR-based collateral-cleavage activity correlates with target concentration.

Cas12's non-specific cleavage can be leveraged to cleave reporters upon target recognition, allowing for the design of sensitive and specific diagnostics using Cas 12, including single nucleotide variants, detection based on rRNA sequences, screening for drug resistance, monitoring microbe outbreaks, genetic perturbations, and screening of environmental samples, as described, for example, in Broughton et al. 2020. CRISPR-Cas12-based detection of SARS-CoV-2. Nat. Biotech. 38:870-874, https://doi.org/10.1038/s41587-020-0513-4; Leung et al. 2021. CRISPR-Cas 12-based nucleic acids detection systems. Methods.; S 1046-2023 (21) 00063-3. doi: 10.1016/j.ymeth.2021.02.018; Mahas et al., Viruses. 2021. 13:466, https://doi.org/10.3390/v13030466; Ali et al., 2020. iSCAN: An RT-LAMP-coupled CRISPR-Cas12 module for rapid, sensitive detection of SARS-COV-2Vir. Res. 288:198129. https://doi.org/10.1016/j.virusres.2020.198129; Ramachandran et al., 2020. Electric field-driven microfluidics for rapid CRISPR-based diagnostics and its application to detection of SARS-CoV-2. PNAS Nov. 24, 2020 117 (47) 29518-29525; Mukama et al., An ultrasensitive and specific point—of care CRISPR-Cas 12 based lateral flow biosensor for the rapid detection of nucleic acids. Biosens Bioelectron. 2020 Jul. 1; 159:112143. doi: 10.1016/j. bios.2020.112143; Chen et al., 2018. CRISPR-Cas12a target binding unleashes indiscriminate single-stranded DNase activity. Science. April 27;360 (6387): 436-439. doi: 10.1126/science.aar6245; Kellner et al., 2019. Nat Protoc. 2019 October; 14 (10): 2986-3012. doi: 10.1038/s41596-019-0210-2; Broughton et al., 2020. Rapid Detection of 2019 Novel Coronavirus SARS-COV-2 Using a CRISPR-based DETECTR Lateral Flow Assay. 2020. medRxiv. March 27;2020.03.06.20032334. doi: 10.1101/2020.03.06.20032334; Wu et al. 2021. CRISPR-Cas 12-Based Rapid Authentication of Halal Food. J Agric Food Chem. 2021 Aug. 26. doi: 10.1021/acs.jafc.lc03078; Long et al. 2021. CRISPR/Cas12-Based Ultra-Sensitive and Specific Point-of-Care Detection of HBV. Int J Mol Sci. 2021 May 3;22 (9): 4842. doi: 10.3390/ijms22094842; Curti et al., Viruses. 2021 Mar. 5; 13 (3): 420. doi: 10.3390/v13030420; Li et al., Cell Discovery (2018) 4:20. DOI 10.1038/s41421-018-0028-z; Lucia et al. 2020. An ultrasensitive, rapid, and portable coronavirus SARS-Cov-2 sequence detection method based on CRISPR-Cas12. bioRxiv preprint doi:

https/doi.org/10.1101/2020.02.29.971127; MammothBiosciences. 2020. Broughton et al., available at https://mammoth.bio/wp-content/uploads/2020/04/200423-A-protocol-for-rapid-detection-of-SARS-COV-2-using-CRISPR-diagnostics_3.pdf; East-Seletsky et al., Nat. 538:270, doi: 10.1038/naturel9802; International Pat. Pub. WO2019/233358; WO2019/011022; U.S. Pat. Pub.: 10,337,051; 10,449,4664, 10,253,365; US 2020/0299768; US 2020/0399697; US 2019/0241954; the disclosure of each can be adapted for use with the present invention in view of the description provided herein and each of which is incorporated herein by reference in its entirety.

Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April;22 (4): 359-66. doi: 10.1002/pro.2225.). Homologous proteins may but need not be structurally related, or are only partially structurally related.

A gRNA is a ribonucleic acid sequence capable of forming a CRISPR system with a CRISPR enzyme. Typically, a gRNA has a conserved sequence forming one or more stem-loop structure(s) (e.g., a direct repeat or “DR”) and a spacer sequence, e.g., a guide sequence. Generally, the CRISPR nuclease (CRISPR enzyme) binds to the DR in the “CRISPR complex” or “complex”, and the spacer sequence comprises a sequence that is complementary to the target nucleic acid. In some instances, complexing of the gRNA “triggers” a nuclease activity of the CRISPR/nuclease gRNA complex. In preferred instances, “triggering” of a nuclease activity of the CRISPR/nuclease gRNA system also requires a presence of a target nucleic acid. A target nucleic acid is one that hybridizes to a direct repeat with sufficient homology to trigger nuclease activation (e.g., greater than 80%). The instant disclosure contemplates systems, reactions mixtures, and methods where the triggering of the nuclease activity by a target nucleic acid activates an enzyme cascade that amplified a signal from the target nucleic acid.

A gRNA can comprise one or more direct repeat sequences, which can form secondary and/or tertiary stem-loop structures. In general, a “direct repeat sequence” includes any sequence that has sufficient sequence to promote formation of a CRISPR system including the gRNA and a nucleic acid-guided nuclease CRISPR enzyme. Sufficient structure within the direct repeat sequence to promote formation of a CRISPR system may include a degree of complementarity along the length of two sequence regions within the scaffold sequence, such as one or two sequence regions involved in forming a secondary structure. In some aspects, the disclosure contemplates a plurality of nucleic acid aptamers that can be cleaved by an activated CRISPR/nuclease complex including the CRISPR enzyme, gRNA, and target polynucleotide sequence. Such activation triggers an enzymatic cascade. With target present, Cas/guideRNA system becomes activated and collaterally cleaves, indiscriminately, nucleic acids in the solution, including the aptamer. Once aptamer gets cleaved, the second enzyme, e.g., reporter enzyme (signal enzyme) employed by the system, e.g., a peroxidase, an oxidase, a phosphatase, a polymerase, or a second CRISPR enzyme, becomes free of the aptamer. The reporting enzyme activity can directly or indirectly generate a detectable signal if a suitable substrate exists in the system.

A gRNA can be compatible with a nucleic acid-guided nuclease (CRISPR enzyme) when the two elements can form a functional CRISPR nuclease complex capable of cleaving a nucleic acid sequence specifically (i.e., targeted activity) and/or non-specifically (i.e., collateral activity) in the presence of a target nucleic acid. In some instances, a gRNA complexed with a compatible nucleic acid-guided nuclease can hybridize with a target sequence, thereby directing the nuclease to indiscriminately cleave all surrounding nucleic acid sequences, i.e., collateral activity. In these cases, the CRISPR nuclease can cleave not only the target sequence, but it may indiscriminately cleave its surrounding nucleic acids despite of their sequence. The invention contemplates reaction schemes for reactions mixtures where the aforementioned collateral activity cleaves a nucleic acid aptamer, thereby freeing a reporter enzyme from the aptamer regulation.

A gRNA can comprise a spacer sequence. A spacer sequence is a polynucleotide sequence having sufficient complementarity with a target polynucleotide sequence to hybridize with the target sequence. The degree of complementarity between a spacer sequence and its corresponding target sequence, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences. In some embodiments, a spacer sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a spacer sequence is less than about 75, 50, 45, 40, 35, 30, 25, 20 nucleotides in length. Preferably the spacer sequence is 10-40 nucleotides long. The spacer sequence can be 15-25 nucleotides in length. The spacer sequence can be ˜15 nucleotides in length. The spacer sequence can be 16 nucleotides in length. The spacer sequence can be 17 nucleotides in length. The spacer sequence can be 18 nucleotides in length. The spacer sequence can be 19 nucleotides in length. The spacer sequence can be 20 nucleotides in length. Non-limiting examples of putative spacer sequences considered by the invention include SEQ ID NOs: 274-366. Certain exemplary gRNAs described herein are tested with specific spacer sequences (e.g., FLU A target), see SEQ ID NOs 94-279. It is to be understood however, that one of ordinary skill in the art, looking at certain tested gRNAs is readily able to identify the direct repeat sequence and distinguish it from the spacer sequence.

A direct repeat sequence of a subject gRNA can comprise a secondary structure and/or a tertiary structure. In some cases, binding kinetics of a gRNA to a nucleic acid-guided nuclease is determined in part by secondary structures within the direct repeat sequence. In some cases, binding kinetics of a gRNA to a nucleic acid-guided nuclease is determined in part by nucleic acid sequence with the scaffold sequence. In CRISPR-based diagnostics, quantification through comparison with a standard curve can be achieved within the picomolar-to-micromolar range (10⁻¹² M-10⁻⁶ M), where CRISPR-based collateral-cleavage activity correlates with target concentration.

In some aspects, the invention provides reaction cascade systems for supporting detection of a target nucleic acid by a CRISPR system with a gRNA comprising a direct repeat sequence and a spacer designed to hybridize (i.e., “target”) a specific target polynucleotide sequence; after hybridization, the disclosure contemplates that activation of the CIRSPR system will lead to cleavage of various aptamers that were previously interacting with a reporter enzyme. The disclosure also contemplates that the reaction mix comprises substrates for such reporter enzymes. The reaction mixtures, methods, kits and systems contemplated by the disclosure include ones where the spacer region of the CRISPR-systems comprises a spacer designed to hybridize to one or more regions of a target selected from the group consisting of: a coronavirus, an Ebola virus, measles, SARS, Chikungunya virus, Marburg, MERS, Dengue, Lassa, influenza, rhabdovirus, HIV, a hepatitis virus (including hepatitis A, B, C, D, or E), an influenza virus (including an influenza A or influenza B), a human respiratory syncytial virus, Sudan ebola virus, Bundibugyo virus, Tai Forest ebola virus, Reston ebola virus, Achimota virus, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bomavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Boma disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human mastadenovirus A-G, Human papillomavirus, Human parainfluenza virus 1-4, Human paraechovirus, Human picomavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozemoe, MSSI2Y225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picomaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bomavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence, or a combination thereof.

### Kits

Any of the reaction mixtures and systems described herein or a combination thereof can be presented as a combination kit. As used herein, the terms “kit components” and “kits” refers to the compounds, compositions, formulations, particles, cells and any additional components that are used to package, sell, market, deliver, and/or administer the combination of reagents or components contained therein. Such additional components include, but are not limited to, packaging, microfluidic devices, tubes, syringes, blister packages, dipsticks, substrates, bottles, and the like. The separate kit components can be contained in a single package or in separate packages within the kit. For instance, in one embodiment the disclosure contemplates a kit with an additional container for extracting nucleic acids from debris or cells of a sample (e.g., enriching nucleic acids from a nasal swab or saliva sample)

In some aspects, the disclosure provides kits having therein tubes with reaction mixtures and enzyme systems that provide functional complexes with nuclease activity once a target is present. In addition, the aforementioned tubes include a reporter enzyme (i.e., a signaling molecule) regulated by a nucleic acid aptamer. A CRISPR reaction mixture of the disclosure typically contains a nucleic acid-guided nuclease (CRISPR nuclease), a gRNA, a complex of a nucleic acid aptamer and a reporter enzyme, and a substrate for the reporter enzyme. Optionally, in any of the reaction mixtures, methods, or other embodiments disclosed herein, depending on the reporter enzyme system selected, any co-factors (e.g., divalent cations such as Mg2) that may be required for activation of one or more of the CRISPR enzyme and/or the reporter enzyme may be present. The reaction mixture may comprise a substrate for the enzyme, such as an RNA or DNA molecule.

Target nucleic acid testing kits may comprise one or more systems of the disclosure, including containers configured to collect a biological sample, including a sputum or nasal swab collection tube for optionally extracting nucleic acids from cells and/or debris. The kits may contain, e.g., blot pads and additional components. Kits may comprise instructions for use of each component.

In some aspects, the disclosure provides kits with one or more components. One component of a kit of the disclosure is a reaction mixture described herein. Each kit can comprise a reaction mixture packaged into at least one tube. The tube may be transparent to facilitated a read-out of a signal provided by a substrate for the reporter enzyme.

A reaction mixture(s) of the disclosure can be package in any suitable tube. In some cases, each tube comprises from 50±5 microliters to 250±5 microliters, from 50±5 microliters to 300±5 microliters, from 50±5 microliters to 350±5 microliters, from 50±5 microliters to 400±5 microliters, from 50±5 microliters to 450±5 microliters, from 50±5 microliters to 500±5 microliters, from 50±5 microliters to 550±5 microliters, from 50±5 microliters to 600±5 microliters, from 50±5 microliters to 650±5 microliters, from 50±5 microliters to 700±5 microliters, from 50±5 microliters to 750±5 microliters, from 50±5 microliters to 800±5 microliters, from 50±5 microliters to 850±5 microliters, from 50±5 microliters to 900±5 microliters, from 50±5 microliters to 950±5 microliters, from 50±5 microliters to 1,000±5 microliters, from 50±5 microliters to 1,050±5 microliters, from 50±5 microliters to 1,100±5 microliters, from 50±5 microliters to 1,150±5 microliters, from 50±5 microliters to 1,200±5 microliters, from 50±5 microliters to 1,250±5 microliters, from 50±5 microliters to 1,300±5 microliters, from 50±5 microliters to 1,350±5 microliters, from 50±5 microliters to 1,400±5 microliters, from 50±5 microliters to 1,450±5 microliters, from 50±5 microliters to 1,500±5 microliters, from 50±5 microliters to 1,550±5 microliters, from 50±5 microliters to 1,600±5 microliters, from 50±5 microliters to 1,650±5 microliters, from 50±5 microliters to 1,700±5 microliters, from 50±5 microliters to 1,750±5 microliters, from 50±5 microliters to 1,800±5 microliters, from 50±5 microliters to 1,850±5 microliters, from 50±5 microliters to 1,900±5 microliters, from 50±5 microliters to 1,950±5 microliters, from 50±5 microliters to 2,000±5 microliters of reaction mixture.

In some embodiments, a kit of the disclosure also includes instructions printed on or otherwise contained in a tangible medium of expression. The instructions can provide information regarding the content of the compounds, compositions, formulations, particles, cells, devices, described herein or a combination thereof contained therein, safety information regarding the content of the compounds, compositions, formulations, particles, devices, and cells described herein or a combination thereof contained therein, information regarding the dosages, working amounts, indications for use, and/or recommended treatment regimen(s) for the compound(s) formulations, devices, and combinations thereof contained therein. In some embodiments, the instructions can provide directions for sample collection, sample preparation, and/or use of the compounds, compositions, formulations, particles, devices and cells described herein or a combination thereof. In some embodiments, the instructions can be specific to the target(s) being detected by a CRISPR effector detection system. In some embodiments, the instructions are specific to detecting a viral target, such as a viral polynucleotide. Exemplary virus that can be detected by the kits described herein are described elsewhere herein. In some embodiments, the viral target is FLUA, FLUB, RSV, or SARS-Cov-2.

### Methods

All of the functionalities described in connection with the systems, kits, and methods described herein are intended to be applicable to detection of at least one target polynucleotide sequence from a sample. Typically, the target polynucleotide sequence is indicative of the presence or amount of a pathogen, e.g., one or more of each of viral nucleic acids, mammalian nucleic acids, and bacterial nucleic acids.

In certain implementations of this method, the reaction mixtures and enzyme systems described herein are configured for the detection of one or more target polynucleotide sequences indicative of the presence and/or amount of one or more of SARS-COV-2, influenza A, influenza B, Human Respiratory Syncytial Virus (RSV). In other cases, the reaction mixtures and enzyme systems described herein are configured for the detection of one or more of SARS-COV-2, influenza A, influenza B, Human Respiratory Syncytial Virus (RSV), adenovirus, coronavirus 229E, coronavirus HKU1, coronavirus NL63, human metapneumovirus, human rhinovirus/enterovirus, parainfluenza virus 1, parainfluenza virus 2, parainfluenza virus 3, parainfluenza virus 4.

### NON-LIMITING WORKING EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. For instance, certain master mixes can in principle be combined; certain enzymes can be replaced by others with similar functionality; certain buffers can be modified depending on the enzyme to be implemented; the number of segments can be altered (e.g., duplicate segments could be implemented in certain configurations of the device and/or certain segments could in principle be combined) without altering the spirit of the invention. The present aspects are, therefore, to be considered in all respects as illustrative and not restrictive.

### Example 1-Reaction Mixtures Comprising a Cas12 CRISPR system, a DNA polymerase, an inhibiting aptamer to the DNA polymerase, and substrates for nucleic acid amplification

### 1.1 Reaction Mixture: DNA Polymerase Inhibiting Aptamer Complex

A reaction mixture is prepared and added into individual single tube(s). The reaction mixture comprises a CRISPR system with a gRNA designed for the detection of a target nucleic acid sequence, FluA. On this particular implementation, a reaction mixture is prepared as follows:

Reaction Mixture 1 (DNA Polymerase + Inhibiting Aptamer)

| Material | Stock | | Conc in | |
|---|---|---|---|---|
| Description | Concentration | | Reaction Mixture | |
| Deionized Water | | | | |
| DNA Polymerase Enzyme ZO5 | 200 | U/μL | 26.93 | U/μL |
| Z05 NTQ aptamer | 200 | mM | 24.62 | mM |
| PCR primers and probes | 10 | mM | 1 | mM |
| dATP | 100 | mM | 3.00 | mM |
| dCTP | 100 | mM | 3.00 | mM |
| dGTP | 100 | mM | 3.00 | mM |
| dUTP | 100 | mM | 3.00 | mM |
| Lba Cas12 | 10000 | nM | 100.00 | nM |
| gRNA (targeting FluA) | 10000 | nM | 150.00 | nM |
| Tris-HCl pH 7.0 | 100 | mM | 18.67 | mM |
| Tween-20 | 10 | % (v/v) | 0.06 | % (v/v) |

The aforementioned system contains Cas 12 and the gRNA designed for the detection of a target nucleic acid sequence, FluA, DNA polymerase as a reporting enzyme (i.e., Roche Z05 enzyme), an inhibiting aptamer for the reporting enzyme (i.e., Z05 NTQ aptamer), and nucleic acid amplification reaction mix (such as PCR TaqMan reaction mix for fluorescence readout). In absence of target pathogen, the inhibiting aptamer binds to DNA polymerase, resulting in inhibited polymerase activity and low fluorescence readout signal. In presence of target pathogen, Cas12 system is activated and cleaves the inhibiting aptamer, resulting in released/increased polymerase activity.

Each reaction mixture is packaged in a single assay tube, which contains all reagents for a single test for detecting a pre-determined target nucleic acid.

### 1.2 Methods of Detecting a Target Sequence with a Reaction Mixture Comprising a DNA Polymerase Inhibiting Aptamer Complex

A target pathogen is detected by contacting a biological sample with the reaction mixture. Presence of the target pathogen is confirmed based on a positive correlation of target vs signal.

### Example 2-Reaction Mixtures Comprising a Cas12 CRISPR system, a peroxidase, an Activiting aptamer for the peroxidase, and a peroxidase substrate

### 2.1 Reaction Mixture: Peroxidase and Activating Aptamer

A reaction mixture is prepared and added into individual single tube(s). The reaction mixture comprises a CRISPR system with a gRNA designed for the detection of a target nucleic acid sequence, FluA. On this particular implementation, a reaction mixture is prepared as follows:

Reaction Mixture 2 (Peroxidase + Activating Aptamer)

| | | | Conc in | |
|---|---|---|---|---|
| Material | Stock | | Reaction | |
| Description | Concentration | | Mixture | |
| Deionized Water | | | | |
| Peroxidase Enzyme | 200 | U/μL | 20 | U/μL |
| G-quaruplex(G4)-forming DNA aptamer | 200 | mM | 20 | mM |
| (5′-GGGTGGGTTGGGAGGG-3′) | | | | |
| (luminol) peroxidase substrate | 10 | mM | 1 | mM |
| Lba Cas12 | 10000 | nM | 100.00 | nM |
| gRNA (targeting FluA) | 10000 | nM | 150.00 | nM |
| Tris-HCl pH 7.0 | 100 | mM | 18.67 | mM |
| Tween-20 | 10 | % (v/v) | 0.06 | % (v/v) |

The aforementioned system contains Cas12 and the gRNA, a horseradish perxodiase, G-quaruplex (G4)-forming DNA aptamer, a luminol peroxidase substrate for chemiluminescence readout. In absence of target pathogen, G4 forming aptamer upregulates the peroxidase activity, resulting in strong enzyme activity and strong enzymatic readout signal. In presence of target pathogen, Cas12 system is activated and cleaves G4 forming apatmer, resulting in non-enhanced peroxidase activity and weak enzymatic readout signal. The target pathogen detection is based on the negative correlation of target vs signal.

### 2.2 Methods of Detecting a Target Sequence with a Reaction Mixture Comprising a Peroxidase Activating Aptamer Complex

A target pathogen is detected by contacting a biological sample with the reaction mixture. Presence of the target pathogen is confirmed based on a negative correlation of target vs signal.

### Example 3-Reaction Mixtures Comprising a Cas12 CRISPR system, a luciferase, an Inhibiting aptamer for the luciferase, and a luciferase substrate

### 3.1 Reaction Mixture: Luciferase and Inhibiting Aptamer

A reaction mixture is prepared and added into individual single tube(s). The reaction mixture comprises a CRISPR system with a gRNA designed for the detection of a target nucleic acid sequence, FluA. On this particular implementation, a reaction mixture is prepared as follows:

Reaction Mixture 3 (Luciferase + Inhibiting Aptamer)

| | | | Conc in | |
|---|---|---|---|---|
| Material | Stock | | Reaction | |
| Description | Concentration | | Mixture | |
| Deionized Water | | | | |
| Luciferase Enzyme | 200 | U/μL | 20 | U/μL |
| Luciferase inhibiting aptamer | 200 | mM | 20 | mM |
| (5′ ATAGTCTATCGATCAATTGGGTCTGGGTCCGTGCCGGCG | | | | |
| TGGTCTTGTAGATAGCAAGTGTATTCA-3′) | | | | |
| Luciferase substrate | 10 | mM | 1 | mM |
| Lba Cas12 | 10000 | nM | 100.00 | mM |
| gRNA (targeting FluA) | 10000 | nM | 150.00 | nM |
| Tris-HCl pH 7.0 | 100 | mM | 18.67 | mM |
| Tween-20 | 10 | % (v/v) | 0.06 | % (v/v) |

### 3.2 Methods of Detecting a Target Sequence with a Reaction Mixture Comprising a Luciferase Inhibiting Aptamer Complex

A target pathogen is detected by contacting a biological sample with the reaction mixture. Presence of the target pathogen is confirmed based on a positive correlation of target vs signal.

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

1. A reaction mix comprising:
a) a CRISPR system comprising: a CRISPR enzyme with nuclease activity and a gRNA, wherein a region of the gRNA is complimentary to a pre-determined target nucleic acid sequence; and
b) a complex of a nucleic acid aptamer and a reporter enzyme, wherein the nucleic acid aptamer in the complex with the reporter enzyme can be cleaved by nuclease activity of the CRISPR enzyme; and
c) a substrate for the reporter enzyme.

2. The reaction mix of claim 1, wherein the nucleic acid aptamer is an inhibitor of the reporter enzyme.

3-8. (canceled)

9. The reaction mix of claim 1, wherein the reporter enzyme is selected from the group consisting of a peroxidase, an oxidase, a phosphatase, a polymerase, or a second CRISPR enzyme.

10-18. (canceled)

19. The reaction mix of claim 1, wherein the substrate for the reporter enzyme provides a fluorescent signal, a chemiluminescence signal, a colorimetric signal, or an electrochemical signal.

20. (canceled)

21. The reaction mix of claim 1, wherein the target nucleic acid sequence is a sequence from one or more of Sars-COV-2, RSV, FluA, FluB, HIV, and human papilloma virus.

22. The reaction mix of claim 1, wherein the CRISPR enzyme comprises trans-cleavage activity.

23. The reaction mix of claim 1, wherein the CRISPR enzyme is a Cas12 enzyme or a Cas13 enzyme.

24-25. (canceled)

26. The reaction mix of claim 1, wherein the reaction mix does not comprise any reagents for nucleic acid amplification.

27. A method, comprising:
i. activating a nuclease activity of a CRISPR enzyme by hybridizing (i) a spacer sequence of a gRNA that is bound with the CRISPR enzyme and (ii) a target polynucleotide sequence; and
ii. modulating an activity of a second enzyme upon a substrate of the second enzyme by cleaving, with the activated CRISPR enzyme, a nucleic acid aptamer bound to the second enzyme.

28. The method of claim 27, wherein the second enzyme is a reporter enzyme and the action of the second enzyme upon the substrate directly or indirectly produces a detectable signal indicative of the presence and/or amount of the target polynucleotide sequence.

29. The method of claim 28, further comprising detecting the detectable signal and determining the presence and/or amount of the target polynucleotide sequence based on the detected detectable signal.

30. The method of claim 27, wherein the method is performed in a liquid mixture, the liquid mixture comprises a biological sample, and the target polynucleotide is present in the biological sample, and further wherein the biological sample comprises a nasal swab, a nasopharyngeal swab, a saliva sample, and/or a sputum sample.

31. The method of claim 27, wherein the method is performed in a liquid mixture, the liquid mixture comprises a biological sample, and the target polynucleotide is present in the biological sample, and further wherein the biological sample comprises a blood sample, e.g., a whole blood sample, a plasma sample, or a serum sample.

32. (canceled)

33. The method of claim 27, wherein the nucleic acid aptamer is an activator of the second enzyme and the step of modulating the activity of the second enzyme comprises decreasing an activity of the second enzyme.

34-35. (canceled)

36. The method of claim 27, wherein the reporter enzyme is selected from the group consisting of a peroxidase, an oxidase, a phosphatase, a polymerase, or a second CRISPR enzyme.

37-45. (canceled)

46. The method of claim 28, wherein the detectable signal is a fluorescence signal, a chemiluminescence signal, a colorimetric signal, or an electrochemical signal.

47. The method of claim 27, wherein the target nucleic acid sequence is a sequence from one or more of Sars-COV-2, RSV, FluA, FluB, HIV, and human papilloma virus.

48. The method of claim 27, wherein the CRISPR enzyme comprises trans-cleavage activity.

49. The method of claim 27, wherein the CRISPR enzyme is a Cas12 enzyme or a Cas 13 enzyme.

50. The method of claim 29, wherein the step of determining the presence and/or amount of the target polynucleotide is performed with a detection limit of no higher than about 10fM.
